SEQUENCE LISTING (28 sequences)

SEQ ID NO: 1 - DNA, 3813 bp, synthetic construct
  misc_feature 1..3813: note = Synthetic: CpG Condon Optimized Polynucleotide Encoding Microdystrophin, AAV Vector Genome
  source 1..3813: mol_type = other DNA
atgctgtggtgggaggaagtggaagattgctacgagcgcgaggacgtgcagaagaaaaccttcaccaaatgggtcaacgcccagttcagcaagttcggcaagcagcacatcgagaacctgttcagcgacctgcaggacggcagacggctgctggatctgctggaaggcctgaccggacagaagctgcccaaagagaagggcagcaccagagtgcacgccctgaacaacgtgaacaaggccctgcgggtgctgcagaacaacaatgtggacctggtgaacattggcagcacagacattgtggatggcaaccacaagctgaccctgggcctgatctggaacatcatcctgcactggcaagtgaagaacgtgatgaagaacatcatggccggcctgcagcagaccaacagcgagaagatcctgctgagctgggtgcgccagagcaccagaaactacccccaagtgaacgtgatcaacttcaccacctcttggagcgacggcctggccctgaatgccctgatccacagccacagacccgacctgtttgactggaacagtgtggtgtgtcagcagagcgccacccagaggctggaacacgccttcaatatcgccagataccagctgggcattgagaagctgctggaccccgaggatgtggacaccacctaccccgacaagaaatccatcctgatgtatatcaccagcctgttccaggtgctgcctcagcaggtgtccatcgaggccatccaggaagtggaaatgctgcccagaccccccaaagtgaccaaagaggaacacttccagctgcaccaccagatgcactactctcagcagatcaccgtgtccctggcccagggctacgagagaaccagcagccccaagccccggttcaagagctacgcctatacccaggccgcctacgtgaccaccagcgaccctaccagaagcccattccccagccagcatctggaagcccccgaggacaagagcttcggcagcagcctgatggaaagcgaagtgaacctggatagataccagaccgccctggaagaggtgctgtcctggctgctgagcgccgaggatacactgcaggctcagggcgagatcagcaatgatgtggaagtggtgaaggaccagttccacacccacgagggctacatgatggacctgacagcccaccagggcagagtgggcaacattctgcagctgggctccaagctgatcggcaccggcaagctgagcgaggacgaagagacagaggtgcaggaacagatgaacctgctgaacagcagatgggagtgcctgagagtggccagcatggaaaagcagagcaacctgcacagctacgtgcccagcacctacctgaccgagatcacccatgtgtcccaggccctgctggaagtggaacagctgctgaacgcccccgatctgtgcgccaaggacttcgaggatctgttcaagcaggaagagagcctgaagaatatcaaggactctctgcagcagtccagcggcagaatcgacatcatccacagcaagaaaacagccgccctgcagtccgccacccccgtggaaagagtgaagctgcaggaagccctgtcccagctggacttccagtgggagaaagtgaacaagatgtacaaggaccggcagggcagatttgaccgcagtgtggaaaagtggaggaggttccactacgacatcaagatcttcaaccagtggctgacagaggccgagcagttcctgagaaagacccagatccccgagaactgggagcacgccaagtacaagtggtatctgaaagaactgcaggatggcattggccagagacagacagtggtgcggacactgaatgccaccggcgaggaaatcatccagcagagcagcaagaccgacgccagtattctgcaggaaaagctgggcagcctgaacctgagatggcaggaagtgtgcaagcagctgtccgaccggaagaagagactggaagaacagagtgaccagtggaagcggctgcatctgtcactgcaggaactgctggtgtggctgcagctgaaggatgatgagctgagcagacaggcccctattggcggcgattttcccgcagtgcagaaacagaacgatgtgcaccgggccttcaagagagagctgaaaacaaaagaaccagtgatcatgagcaccctggaaacagtgcggatctttctgaccgagcagcccctggaaggactggaaaaactgtaccaggaacccagagagctgccccctgaagaacgggcccagaacgtgaccagactgctgaggaagcaggccgaggaagtgaacacagaatgggagaagctgaacctgcactctgctgactggcagaggaagattgatgagacactggaacggctgcaggaactgcaggaggccacagacgagctggacctgaaactgagacaggccgaagtgatcaagggcagctggcagccagtgggcgacctgctgatcgacagcctgcaggatcacctggaaaaagtgaaagccctgagaggcgagattgcccccctgaaagaaaatgtgtcccatgtgaacgacctggcccggcagctgacaacactgggcatccagctgagcccctacaacctgtccacactggaagatctgaacacccggtggaaactgctgcaggtggccgtggaagatagagtgcggcagctgcacgaggcccacagagattttggccctgcctcccagcacttcctgagcacatctgtgcagggcccctgggagagagccatctcccccaacaaggtgccctactacatcaaccacgagacacagaccacctgttgggaccaccccaagatgacagagctgtaccagagcctggccgacctgaacaatgtgaggttcagtgcctacaggaccgccatgaagctgcggagactgcagaaagctctgtgcctggacctgctgtccctgtccgccgcttgtgatgccctggaccagcacaacctgaagcagaacgaccagcccatggatatcctgcagatcatcaactgcctgaccaccatctacgaccgcctggaacaggaacacaacaacctggtgaatgtgcccctgtgtgtggacatgtgcctgaattggctgctgaatgtgtacgacaccggccggacaggccggatcagagtgctgagcttcaagaccggcatcatcagcctgtgcaaggcccacctggaagataagtaccgctacctgttcaaacaggtggccagctccaccggcttttgcgaccagagaaggctgggcctgctgctgcacgacagcatccagatccctagacagctgggcgaggtggcctcttttggcggcagcaatattgagcctagtgtgcggagctgcttccagtttgccaacaacaagcccgagattgaggccgccctgttcctggactggatgcggctggaaccccagagcatggtgtggctgcctgtgctgcatagagtggccgctgccgagacagccaagcaccaggccaagtgcaacatctgcaaagagtgccccatcatcggcttccggtacagaagcctgaagcacttcaactacgatatctgccagagctgctttttcagcggacgggtggccaagggccacaaaatgcactaccccatggtggaatactgcacccccaccacctccggggaggatgtgcgggattttgccaaggtgctgaaaaacaagttccggaccaagcgctactttgccaaacacccccggatgggctatctgcccgtgcagacagtgctggaaggcgacaacatggaaaccgacaccatgtag

SEQ ID NO: 2 - protein, 1270 aa, synthetic construct
  REGION 1..1270: note = Synthetic: microdystrophin protein
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 3 - DNA, 447 bp, synthetic construct
  misc_feature 1..447: note = Synthetic: muscle-specific control element comprises a CK8 promoter
  source 1..447: mol_type = other DNA
tagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagcca

SEQ ID NO: 4 - DNA, 577 bp, synthetic construct
  misc_feature 1..577: note = Synthetic: CK8e promoter
  source 1..577: mol_type = other DNA
tagactagcatgctgcccatgtaaggaggcaaggcctggggacacccgagatgcctggttataattaacccagacatgtggctgcccccccccccccaacacctgctgcctctaaaaataaccctgcatgtagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagcca

SEQ ID NO: 5 - DNA, 130 bp, synthetic construct
  misc_feature 1..130: note = Synthetic: 130-bp enhancer
  source 1..130: mol_type = other DNA
tagactagcatgctgcccatgtaaggaggcaaggcctggggacacccgagatgcctggttataattaacccagacatgtggctgcccccccccccccaacacctgctgcctctaaaaataaccctgcatg

SEQ ID NO: 6 - DNA, 269 bp, synthetic construct
  misc_feature 1..269: note = Synthetic: 269-bp basal CK8 promoter
  source 1..269: mol_type = other DNA
ccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctc

SEQ ID NO: 7 - DNA, 48 bp, synthetic construct
  misc_feature 1..48: note = Synthetic: UTR 48 bp MCK Exon 1
  source 1..48: mol_type = other DNA
attctaccaccacctccacagcacagacagacactcaggagccagcca

SEQ ID NO: 8 - DNA, 48 bp, synthetic construct
  misc_feature 1..48: note = Synthetic: polyA signal sequence
  source 1..48: mol_type = other DNA
aataaaagatccttattttcattggatctgtgtgttggttttttgtgt

SEQ ID NO: 9 - DNA, 197 bp, synthetic construct
  misc_feature 1..197: note = Synthetic: SV40 polyA signal sequence
  source 1..197: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttta

SEQ ID NO: 10 - DNA, 230 bp, synthetic construct
  misc_feature 1..230: note = Synthetic: bGH polyA signal sequence
  source 1..230: mol_type = other DNA
gtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 11 - DNA, 127 bp, synthetic construct
  misc_feature 1..127: note = Synthetic: rBG polyA signal sequence
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 12 - DNA, 130 bp, synthetic construct
  misc_feature 1..130: note = Synthetic: 5' ITR
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 13 - DNA, 130 bp, synthetic construct
  misc_feature 1..130: note = Synthetic: 3' ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 129 bp, synthetic construct
  misc_feature 1..129: note = Synthetic: intron
  source 1..129: mol_type = other DNA
gtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 4639 bp, synthetic construct
  misc_feature 1..4639: note = Synthetic: AAV vector genome
  source 1..4639: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccactttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagaaccatgctgtggtgggaggaagtggaagattgctacgagcgcgaggacgtgcagaagaaaaccttcaccaaatgggtcaacgcccagttcagcaagttcggcaagcagcacatcgagaacctgttcagcgacctgcaggacggcagacggctgctggatctgctggaaggcctgaccggacagaagctgcccaaagagaagggcagcaccagagtgcacgccctgaacaacgtgaacaaggccctgcgggtgctgcagaacaacaatgtggacctggtgaacattggcagcacagacattgtggatggcaaccacaagctgaccctgggcctgatctggaacatcatcctgcactggcaagtgaagaacgtgatgaagaacatcatggccggcctgcagcagaccaacagcgagaagatcctgctgagctgggtgcgccagagcaccagaaactacccccaagtgaacgtgatcaacttcaccacctcttggagcgacggcctggccctgaatgccctgatccacagccacagacccgacctgtttgactggaacagtgtggtgtgtcagcagagcgccacccagaggctggaacacgccttcaatatcgccagataccagctgggcattgagaagctgctggaccccgaggatgtggacaccacctaccccgacaagaaatccatcctgatgtatatcaccagcctgttccaggtgctgcctcagcaggtgtccatcgaggccatccaggaagtggaaatgctgcccagaccccccaaagtgaccaaagaggaacacttccagctgcaccaccagatgcactactctcagcagatcaccgtgtccctggcccagggctacgagagaaccagcagccccaagccccggttcaagagctacgcctatacccaggccgcctacgtgaccaccagcgaccctaccagaagcccattccccagccagcatctggaagcccccgaggacaagagcttcggcagcagcctgatggaaagcgaagtgaacctggatagataccagaccgccctggaagaggtgctgtcctggctgctgagcgccgaggatacactgcaggctcagggcgagatcagcaatgatgtggaagtggtgaaggaccagttccacacccacgagggctacatgatggacctgacagcccaccagggcagagtgggcaacattctgcagctgggctccaagctgatcggcaccggcaagctgagcgaggacgaagagacagaggtgcaggaacagatgaacctgctgaacagcagatgggagtgcctgagagtggccagcatggaaaagcagagcaacctgcacagctacgtgcccagcacctacctgaccgagatcacccatgtgtcccaggccctgctggaagtggaacagctgctgaacgcccccgatctgtgcgccaaggacttcgaggatctgttcaagcaggaagagagcctgaagaatatcaaggactctctgcagcagtccagcggcagaatcgacatcatccacagcaagaaaacagccgccctgcagtccgccacccccgtggaaagagtgaagctgcaggaagccctgtcccagctggacttccagtgggagaaagtgaacaagatgtacaaggaccggcagggcagatttgaccgcagtgtggaaaagtggaggaggttccactacgacatcaagatcttcaaccagtggctgacagaggccgagcagttcctgagaaagacccagatccccgagaactgggagcacgccaagtacaagtggtatctgaaagaactgcaggatggcattggccagagacagacagtggtgcggacactgaatgccaccggcgaggaaatcatccagcagagcagcaagaccgacgccagtattctgcaggaaaagctgggcagcctgaacctgagatggcaggaagtgtgcaagcagctgtccgaccggaagaagagactggaagaacagagtgaccagtggaagcggctgcatctgtcactgcaggaactgctggtgtggctgcagctgaaggatgatgagctgagcagacaggcccctattggcggcgattttcccgcagtgcagaaacagaacgatgtgcaccgggccttcaagagagagctgaaaacaaaagaaccagtgatcatgagcaccctggaaacagtgcggatctttctgaccgagcagcccctggaaggactggaaaaactgtaccaggaacccagagagctgccccctgaagaacgggcccagaacgtgaccagactgctgaggaagcaggccgaggaagtgaacacagaatgggagaagctgaacctgcactctgctgactggcagaggaagattgatgagacactggaacggctgcaggaactgcaggaggccacagacgagctggacctgaaactgagacaggccgaagtgatcaagggcagctggcagccagtgggcgacctgctgatcgacagcctgcaggatcacctggaaaaagtgaaagccctgagaggcgagattgcccccctgaaagaaaatgtgtcccatgtgaacgacctggcccggcagctgacaacactgggcatccagctgagcccctacaacctgtccacactggaagatctgaacacccggtggaaactgctgcaggtggccgtggaagatagagtgcggcagctgcacgaggcccacagagattttggccctgcctcccagcacttcctgagcacatctgtgcagggcccctgggagagagccatctcccccaacaaggtgccctactacatcaaccacgagacacagaccacctgttgggaccaccccaagatgacagagctgtaccagagcctggccgacctgaacaatgtgaggttcagtgcctacaggaccgccatgaagctgcggagactgcagaaagctctgtgcctggacctgctgtccctgtccgccgcttgtgatgccctggaccagcacaacctgaagcagaacgaccagcccatggatatcctgcagatcatcaactgcctgaccaccatctacgaccgcctggaacaggaacacaacaacctggtgaatgtgcccctgtgtgtggacatgtgcctgaattggctgctgaatgtgtacgacaccggccggacaggccggatcagagtgctgagcttcaagaccggcatcatcagcctgtgcaaggcccacctggaagataagtaccgctacctgttcaaacaggtggccagctccaccggcttttgcgaccagagaaggctgggcctgctgctgcacgacagcatccagatccctagacagctgggcgaggtggcctcttttggcggcagcaatattgagcctagtgtgcggagctgcttccagtttgccaacaacaagcccgagattgaggccgccctgttcctggactggatgcggctggaaccccagagcatggtgtggctgcctgtgctgcatagagtggccgctgccgagacagccaagcaccaggccaagtgcaacatctgcaaagagtgccccatcatcggcttccggtacagaagcctgaagcacttcaactacgatatctgccagagctgctttttcagcggacgggtggccaagggccacaaaatgcactaccccatggtggaatactgcacccccaccacctccggggaggatgtgcgggattttgccaaggtgctgaaaaacaagttccggaccaagcgctactttgccaaacacccccggatgggctatctgcccgtgcagacagtgctggaaggcgacaacatggaaaccgacaccatgtaggaagtcttttaataaaagatccttattttcattggatctgtgtgttggttttttgtgtcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 50 bp, synthetic construct
  misc_feature 1..50: note = Synthetic: UTR 50 bp MCK Exon 1
  source 1..50: mol_type = other DNA
attctaccaccacctccacagcacagacagacactcaggagccagccagc

SEQ ID NO: 17 - DNA, 206 bp, synthetic construct
  misc_feature 1..206: note = Synthetic: muscle-specific control element
  source 1..206: mol_type = other DNA
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgg

SEQ ID NO: 18 - DNA, 358 bp, synthetic construct
  misc_feature 1..358: note = Synthetic: muscle-specific control element
  source 1..358: mol_type = other DNA
gctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 19 - DNA, 11 bp, synthetic construct
  misc_feature 1..11: note = Synthetic: 5' heterologous sequence
  source 1..11: mol_type = other DNA
cctgcaggcag

SEQ ID NO: 20 - protein, 736 aa, synthetic construct
  REGION 1..736: note = Synthetic: AAV9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 743 aa, synthetic construct
  REGION 1..743: note = Synthetic: VP1 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLGLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 10 bp, synthetic construct
  misc_feature 1..10: note = Synthetic: 5' heterologous sequence
  source 1..10: mol_type = other DNA
ttaattaagg

SEQ ID NO: 23 - DNA, 11 bp, synthetic construct
  misc_feature 1..11: note = Synthetic: modified 5' ITR sequence
  source 1..11: mol_type = other DNA
aaagcccgggc

SEQ ID NO: 24 - DNA, 141 bp, synthetic construct
  misc_feature 1..141: note = Synthetic: 5' ITR
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 25 - DNA, 130 bp, synthetic construct
  misc_feature 1..130: note = Synthetic: 5' ITR
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 26 - DNA, 140 bp, synthetic construct
  misc_feature 1..140: note = Synthetic: 5' ITR
  source 1..140: mol_type = other DNA
ttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 27 - DNA, 141 bp, synthetic construct
  misc_feature 1..141: note = Synthetic: 3' ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 140 bp, synthetic construct
  misc_feature 1..140: note = Synthetic: 3' ITR
  source 1..140: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

(19) United States
(12) Patent Application Publication
(10) Pub. No.: US 2024/0350674 A1
(43) Pub. Date: Oct. 24, 2024

(54) TREATMENT OF MUSCULAR DYSTROPHY

(71) Applicant: Solid Biosciences Inc., Charlestown, MA (US)

(72) Inventors: Kristy Jean Brown, Rockville, MD (US); Jennifer Green, Cambridge, MA (US)

(21) Appl. No.: 18/682,359

(22) PCT Filed: Aug. 11, 2022
(86) PCT No.: PCT/US2022/040030

§ 371 Date: Feb. 8, 2024

Related U.S. Application Data
(60) Provisional application No. 63/231,720, filed on Aug. 11, 2021.

(51) Int. Cl. A61K 48/00; C07K 14/47; C12N 15/86

(52) U.S. Cl. CPC A61K 48/0058; A61K 48/0033; A61K 48/0066; C07K 14/4707; C12N 15/86; C12N 2750/14143; C12N 2750/14145

(57) ABSTRACT

The invention described herein provides a microdystrophin-encoding, codon optimized polynucleotide with reduced CPG island, and use thereof in the treatment of muscular dystrophy such as DMD/BMD.

### REFERENCE TO RELATED APPLICATION

This application claims priority to and the benefit of the filing date of U.S. Provisional Patent Application No. 63/231,720, filed on Aug. 11, 2021, the entire content of which including any drawings and sequence listing are incorporated herein by reference.

### BACKGROUND OF THE INVENTION

Muscular dystrophy (MD) is a group of diseases that cause progressive weakness and loss of muscle mass. In muscular dystrophy, abnormal genes (mutant genes) produce no functional wild-type proteins needed to form healthy muscle.

Muscular dystrophies have serious debilitating impacts on quality of life of affected patients. Duchenne type muscular dystrophy (DMD) is one of the most devastating muscle diseases affecting 1 in 5,000 newborn males. It is the most well-characterized muscular dystrophy, resulting from mutations in genes encoding members of the dystrophin-associated protein complex (DAPC). These MDs result from membrane fragility associated with the loss of sarcolemmal-cytoskeleton tethering by the DAPC.

Specifically, DMD is caused by mutations in the DMD gene, leading to reductions in DMD mRNA and the absence of dystrophin or functional dystrophin, a 427 kDa sarcolemmal protein associated with the dystrophin-associated protein complex (DAPC) (Hoffman et al., Cell 51(6):919-928, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction, and protect against contraction-induced damage.

Loss of dystrophin as a result of DMD gene mutations disrupts the dystrophin glycoprotein complex, leading to increased muscle membrane fragility. A cascade of events including influx of calcium into the sarcoplasm, activation of proteases and proinflammatory cytokines, and mitochondrial dysfunction results in progressive muscle degeneration. In addition, displacement of neuronal nitric oxide synthase (nNOS) contributes to tissue ischemia, increased oxidative stress, and reparative failure. Disease progression is characterized by increasing muscle necrosis, fibrosis, and fatty tissue replacement and a greater degree of fiber size variation seen in subsequent muscle biopsies.

Currently there is no cure for DMD. The standard of care includes administering corticosteroids (such as prednisone or deflazacort) to stabilize muscle strength and function, prolonging independent ambulation, and delaying scoliosis and cardiomyopathy; bisphosphonates; and denosumab and recombinant parathyroid hormones.

With the advent of gene therapy, research and clinical trials for DMD treatment has focused on gene replacement or other genetic therapies aimed to at least partially restore dystrophin function. These include supplying a functional copy of the dystrophin gene, such as a dystrophin minigene, or repairing a defective dystrophin gene product by exon skipping and nonsense mutation suppression.

Adeno-associated virus (AAV) is a replication-deficient parvovirus, the single-stranded DNA genome of which is about 4.7 kb in length, including 145 nucleotide inverted terminal repeat (ITRs).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells, allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids, which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther 8:659-669 (1997); Kessler et al., Proc Nat. Acad Sc. U.S.A. 93:14082-14087 (1996); and Xiao et al., J Virol 70: 8098-8108 (1996). See also, Chao et al., Mol Ther 2:619-623 (2000) and Chao et al., Mol Ther 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci U.S.A. 94: 5804-5809 (1997) and Murphy et al., Proc Natl Acad Sci U.S.A. 94: 13921-13926 (1997). Moreover, Lewis et al., J Virol 76: 8769-8775 (2002) demonstrated that skeletal myofibers possess the necessary cellular factors for correct antibody glycosylation, folding, and secretion, indicating that muscle is capable of stable expression of secreted protein therapeutics.

In order to optimize expression level of the AAV-delivered microdystrophin construct, the microdystrophin coding sequence may be codon optimized for optimal expression in target cells, such as muscle cells. However, many conventional codon optimization processes inadvertently introduces CpG motifs to the codon-optimized coding sequence. Methylated CpG motifs or CpG islands tends to suppress gene expression, while unmethylated CpG motifs tends to trigger high immunogenicity against the viral construct.

### SUMMARY OF THE INVENTION

One aspect of the invention provides a polynucleotide encoding the microdystrophin of SEQ ID NO: 2, said polynucleotide comprising the nucleotide sequence of SEQ ID NO: 1, or a nucleotide sequence at least 70%, 75%, 80%, 85%, 90%, 95%, 96%, 97%, 98%, 99%, 99.2%, 99.4%, 99.6%, 99.8%, or 99.9% identical thereto.

In certain embodiments, the polynucleotide is identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides.

In certain embodiments, the polynucleotide substantially lacks CpG islands (e.g., no more than 1, 2, 3, 4, 5, 6, 7, 8, 9, or 10 CpG islands based on EMBOSS Cpgplot analysis).

In certain embodiments, the polynucleotide comprises, consists essentially of, or consists of a nucleotide sequence at least 95% identical to SEQ ID NO: 1.

In certain embodiments, the polynucleotide comprises, consists essentially of, or consists of a nucleotide sequence at least 97% identical to SEQ ID NO: 1.

In certain embodiments, the polynucleotide comprises, consists essentially of, or consists of a nucleotide sequence at least 99% identical to SEQ ID NO: 1.

In certain embodiments, the polynucleotide comprises the nucleotide sequence of SEQ ID NO: 1.

In certain embodiments, the polynucleotide consists of the nucleotide sequence of SEQ ID NO: 1.

Another aspect of the invention provides an adeno associated virus (AAV) vector genome, comprising the polynucleotide of the invention, wherein the AAV vector genome is capable of being packaged inside an AAV capsid.

Another aspect of the invention provides a recombinant adeno associated viral (rAAV) particle, comprising an AAV capsid, and an AAV vector genome comprising the polynucleotide of the invention, wherein the AAV vector genome is encapsidated within the AAV capsid.

In certain embodiments, in the AAV vector genome or the rAAV viral particle of the invention, the polynucleotide is operably linked to a transcriptional regulatory element.

In certain embodiments, in the AAV vector genome or the rAAV viral particle of the invention, the transcriptional regulatory element comprises a promoter.

In certain embodiments, in the AAV vector genome or the rAAV viral particle of the invention, the promoter is a muscle-specific promoter.

In certain embodiments, in the AAV vector genome or the rAAV viral particle of the invention, the muscle-specific promoter is CK8 promoter, cardiac troponin T (cTnT) promoter, CK7 promoter, CK9 promoter, truncated MCK (tMCK), myosin heavy chain (MHC) promoter, hybrid α-myosin heavy chain enhancer-/MCK enhancer-promoter (MHCK7), a muscle specific creatine kinase (MCK) promoter, human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

In certain embodiments, in the AAV vector genome or the rAAV viral particle of the invention, the muscle-specific promoter is a CK8 promoter; optionally, said CK8 promoter comprises the nucleotide sequence of SEQ ID NO: 3 or 4.

In certain embodiments, in the AAV vector genome or the rAAV viral particle of the invention, the vector genome further comprises a polyadenylation signal sequence, such as the polyA signal sequence of SEQ ID NO: 8.

In certain embodiments, in the AAV vector genome or the rAAV viral particle of the invention, the polyadenylation signal sequence comprises an SV40 polyadenylation signal sequence (e.g., SEQ ID NO: 9), a bovine growth hormone (bGH) polyadenylation signal sequence (e.g., SEQ ID NO: 10), or a rabbit beta globin (rBG) polyadenylation signal sequence (e.g., SEQ ID NO: 11).

In certain embodiments, in the AAV vector genome or the rAAV viral particle of the invention, the vector genome further comprises a 3′ ITR sequence, such as an AAV2 3′ ITR sequence.

In certain embodiments, in the AAV vector genome or the rAAV viral particle of the invention, the vector genome further comprises a 5′ ITR sequence, such as an AAV2 5′ ITR sequence.

In certain embodiments, in the AAV vector genome or the rAAV viral particle of the invention, the 5′ ITR sequence, and/or the 3′ ITR sequence comprise or are SEQ ID NOs: 12 and 13, respectively.

In certain embodiments, in the AAV vector genome or the rAAV viral particle of the invention, the vector genome further comprises an intron and/or an exon sequence that enhances expression of the microdystrophin.

In certain embodiments, in the AAV vector genome or the rAAV viral particle of the invention, the intron comprises SEQ ID NO: 14.

In certain embodiments, in the AAV vector genome or the rAAV viral particle of the invention, the vector genome further comprises a 5′ UTR sequence, and/or a 3′ UTR sequence.

In certain embodiments, in the AAV vector genome or the rAAV viral particle of the invention, the AAV vector genome or the rAAV viral particle of the invention comprises, consists essentially of, or consists of the nucleotide sequence of SEQ ID NO: 15, or a nucleotide sequence at least 90%, 95%, 96%, 97%, 98%, 99%, 99.5%, 99.7% or 99.9% identical thereto.

In certain embodiments, the capsid of the viral particle is of the serotype of SLB-101, AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, or AAVhu37.

In certain embodiments, the capsid is of the serotype of SLB-101 or AAV9.

Another aspect of the invention provides a recombinant adeno-associated virus (rAAV) viral particle, comprising an SLB-101 or AAV9 capsid, and a vector genome encapsidated therein, wherein said vector genome comprises a polynucleotide sequence encoding the MD5 microdystrophin of SEQ ID NO: 2.

In certain embodiments, the polynucleotide sequence comprises the nucleotide sequence of SEQ ID NO: 1.

In certain embodiments, the polynucleotide sequence comprises a nucleotide sequence at least 95%, 96%, 97%, 98%, 99% identical to SEQ ID NO: 1, and is identical to SEQ ID NO: 1 at each capitalized nucleotides.

In certain embodiments, the vector genome comprises a muscle-specific control element operably linked to the polynucleotide sequence.

In certain embodiments, the muscle-specific control element comprises a CK8 promoter, such as the CK8 promoter of the nucleotide sequence of SEQ ID NO: 3 or 4.

In certain embodiments, the vector genome further comprises a polyadenylation signal sequence, such as a polyA signal sequence comprising SEQ ID NO: 8.

In certain embodiments, the polyadenylation signal sequence comprises an SV40 polyadenylation signal sequence (SEQ ID NO: 9), a bovine growth hormone (bGH) polyadenylation signal sequence (SEQ ID NO: 10), or a rabbit beta globin (rBG) polyadenylation signal sequence (SEQ ID NO: 11).

In certain embodiments, the vector genome further comprises a 3′ ITR sequence, such as SEQ ID NO: 3′ ITR; and a 5′ ITR sequence, such as SEQ ID NO: 5′ ITR.

In certain embodiments, the AAV viral particle of the invention comprises, consists essentially of, or consists of the nucleotide sequence of SEQ ID NO: 15, or a nucleotide sequence at least 90%, 95%, 96%, 97%, 98%, 99%, 99.5%, 99.7% or 99.9% identical thereto.

Another aspect of the invention provides a pharmaceutical composition comprising the polynucleotide of the invention, the rAAV vector genome or the rAAV viral particle of the invention, and a pharmaceutically acceptable carrier.

In certain embodiments, the pharmaceutical composition is suitable or formulated for intravenous, subcutaneous, intramuscular, intradermal, intraperitoneal, or intrathecal administration.

Another aspect of the invention provides a method of treating a muscular dystrophy in a human in need thereof, the method comprising administering to the human a therapeutically effective amount of the polynucleotide of the invention, the rAAV vector genome or the rAAV viral particle of the invention, or the pharmaceutical composition of the invention.

In certain embodiments, the muscular dystrophy is characterized by a loss-of-function a mutation in the dystrophin gene.

In certain embodiments, the muscular dystrophy is Duchenne muscular dystrophy (DMD), Becker muscular dystrophy (BMD), or X-linked dilated cardiomyopathy.

In certain embodiments, the rAAV viral particle is administered at a dose of about 1×10¹² to about 1×10¹⁶ vector genome (vg)/kg, or about 1×10¹³ to about 1×10¹⁵ vector genome (vg)/kg.

Another aspect of the invention provides q host cell comprising the polynucleotide of the invention, or the rAAV vector genome or the rAAV viral particle of the invention.

In certain embodiments, the host cell is a HeLa cell, a Cos7 cell, a HEK293 cell, an A549 cell, a BHK cell, a Vero cell, an RD cell, an HT-1080 cell, an ARPE-19 cell, or a MRC-5 cell.

In certain embodiments, the host cell is a HeLa cell or a 293/293T cell.

It should be understood that any one embodiment of the invention described herein can be combined with any one or more additional embodiments of the invention, including those embodiments described only in the examples or only described in one of the sections above or below, or one aspect of the invention.

### BRIEF DESCRIPTION OF THE DRAWINGS

FIG. 1 shows results output from the EMBOSS Cpgplot online tool, for the native (not codon optimized) human microdystrophin coding sequence encoding SEQ ID NO: 2. One (1) CpG island was identified by the online tool. The parameters chosen: window size=100, minimum length=100, minimum observed=0.6, minimum percentage=50.

FIG. 2 shows results output from the EMBOSS Cpgplot online tool, for a first codon optimized human microdystrophin coding sequence encoding SEQ ID NO: 2. Nine (9) CpG islands were identified by the online tool. Codon optimization was performed using Gene Art. The parameters chosen: window size=100, minimum length=100, minimum observed=0.6, minimum percentage=50.

FIG. 3 shows results output from the EMBOSS Cpgplot online tool, for SEQ ID NO: 1, which results from removing the 9 identified CpG islands in the first codon optimized human microdystrophin coding sequence encoding SEQ ID NO: 2. No (0) CpG islands was identified by the online tool. The parameters chosen: window size=100, minimum length=100, minimum observed=0.6, minimum percentage=50.

FIG. 4 shows results output from the EMBOSS Cpgplot online tool, for a second codon optimized human microdystrophin coding sequence encoding SEQ ID NO: 2. Four (4) CpG islands were identified by the online tool. Codon optimization was performed using GenScript. The parameters chosen: window size=100, minimum length=100, minimum observed=0.6, minimum percentage=50.

FIG. 5 shows results output from the EMBOSS Cpgplot online tool, for a third codon optimized human microdystrophin coding sequence encoding SEQ ID NO: 2. Eleven (11) CpG islands were identified by the online tool. Codon optimization was performed using DNA2.0. The parameters chosen: window size=100, minimum length=100, minimum observed=0.6, minimum percentage=50.

FIG. 6 shows results output from the EMBOSS Cpgplot online tool, for a fourth codon optimized human microdystrophin coding sequence encoding SEQ ID NO: 2. Ten (10) CpG islands were identified by the online tool. Codon optimization was performed using DNA2.0, using the first codon optimized human microdystrophin coding sequence (codon optimized by Gene Art) as input. The parameters chosen: window size=100, minimum length=100, minimum observed=0.6, minimum percentage=50. Thus, successive rounds of codon optimization using different methods did not eliminate CpG islands.

FIG. 7 shows a schematic drawing for the TLR9 assay described in Example 2.

### DETAILED DESCRIPTION OF THE INVENTION

### 1. Overview

The invention described herein provides CpG island reduced or substantially eliminated version of certain codon optimized microdystrophin coding sequences, and use thereof with minimized risk for triggering undesirable host immunity and/or expression silencing.

The invention is partly based on the discovery that certain codon optimized sequences, optimized for optimal expression in mammalian cells, inadvertently introduces CpG motifs or CpG islands, and that such CpG motifs can be substantially reduced or eliminated to avoid triggering undesired host immune responses, while substantially maintaining enhanced expression resulting from codon-optimization.

CpG motifs contain a cytosine triphosphate deoxynucleotide (“C”) followed by a guanine triphosphate deoxynucleotide (“G”). The “p” in between refers to the phosphodiester link between consecutive nucleotides. When these CpG motifs are methylated, they may suppress expression from the coding sequence comprising or adjacent to methylated CpG motifs. On the other hand, when the CpG motifs are unmethylated, they can act as immunostimulants that may induce undesired host immune response.

CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed only in B cells and plasmacytoid dendritic cells (pDCs) in humans and other higher primates. Binding and activation of TLR9 by unmethylated CpG motifs promotes CTL responses to AAV vectors in non-clinical models. Polynucleotides containing unmethylated CpGs have been used as adjuvants in vaccine development to stimulate strong cellular immune responses. Meanwhile, many gene therapy trials using differing codon-modification strategies have resulted in a broad range of CpG content (0- to 5-fold of wild type) in the respective open reading frames (ORFs), and a strong correlation has been found between low CpG content to long-term expression.

Numerous sequences have been shown to stimulate TLR9 with variations in the number and location of CpG dimers, as well as the precise base sequences flanking the CpG dimers. As a result, CpG motifs can be classified roughly as 5 classes or categories based on their sequence, secondary structures, and effect on human peripheral blood mononuclear cells (PBMCs).

For example, using synthetic oligodeoxynucleotides (ODN), class A CpG motif containing ODN has the structural feature of: (1) the presences of a poly G sequence at the 5′ end, the 3′ end, or both; (2) an internal palindrome sequence; (3) GC dinucleotides contained within the internal palindrome; and (4) a partially PS-modified backbone. This class of ODN stimulates the production of large amounts of Type I interferons, the most important one being IFNα, and induced the maturation of plasmacytoid dendritic cells. Class A ODN are also strong activators of NK cells through indirect cytokine signaling.

In contrast, Class B CpG motif containing ODN has the following structural features: (1) one or more 6-mer CpG motif 5′-Pu Py C G Py Pu-3′; (2) a fully phosphorothioated (PS-modified) backbone; and (3) generally 18 to 28 nucleotides in length. Class B ODN (i.e. ODN 2007) are strong stimulators of human B cell and monocyte maturation. They also stimulate the maturation of pDC but to a lesser extent than Class A ODN and very small amounts of IFNα.

There are software or online tools known to one of skill in the art to predict the presence of different classes of CpG motifs that are possible to cause various immune response in the host. For example, the EMBOSS Cpgplot is an online tool at URL ebi.ac.uk slash Tools slash seqstats slash emboss_cpgplot, which requires an input nucleotide sequence. Typical parameters include window size of about 100 nts, minimum length of about 200 nts (which can be adjusted to, e.g., 100, in some embodiments), minimum observed of about 0.6, and minimum percentage of about 50(%). The return will include a number of results, including putative CpG islands or the absence thereof.

By inspecting a codon optimized polynucleotide encoding the microdystrophin of SEQ ID NO: 2, numerous potential CpG motifs were identified, and manually eliminated, while maintaining the ability of the resulting sequence to encode SEQ ID NO: 2.

One exemplary polynucleotide of the invention comprises numerous such nucleotide sequence changes, as described in the section below, as “capitalized nucleotides,” as described herein below. The collection of such capitalized nucleotides constitute a signature for nucleotide sequence changes in SEQ ID NO: 1 to reduce the impact of any CpG motifs. Other polynucleotide of the invention containing the same signature changes, even though they differ from SEQ ID NO: 1 at numerous other nucleotides, e.g., down to about 70% sequence identity, can still encode SEQ ID NO: 2 due to codon degeneracy.

Thus in one aspect, the invention provides a polynucleotide encoding the microdystrophin of SEQ ID NO: 2, said polynucleotide comprising the nucleotide sequence of SEQ ID NO: 1, or a nucleotide sequence at least 70%, 75%, 80%, 85%, 90%, 95%, 96%, 97%, 98%, 99%, 99.2%, 99.4%, 99.6%, 99.8%, or 99.9% identical thereto.

In certain embodiments, the polynucleotide of the invention is identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides.

In certain embodiments, the polynucleotide of the invention substantially lacks CpG islands, e.g., no more than 1, 2, 3, 4, 5, 6, 7, 8, 9, or 10 CpG islands. The presence or absence of CpG motifs or islands can be predicted based on the polynucleotide sequence using art recognized software, such as the EMBOSS Cpgplot online tool.

In certain embodiments, the polynucleotide of the invention comprise, consists essentially of, or consists of a nucleotide sequence at least 95% identical to SEQ ID NO: 1.

In certain embodiments, the polynucleotide of the invention comprise, consists essentially of, or consists of a nucleotide sequence at least 97% identical to SEQ ID NO: 1.

In certain embodiments, the polynucleotide of the invention comprise, consists essentially of, or consists of a nucleotide sequence at least 99% identical to SEQ ID NO: 1.

In certain embodiments, the polynucleotide of the invention comprises, consists essentially of, or consists of the nucleotide sequence of SEQ ID NO: 1.

Another aspect of the invention provides an adeno associated virus (AAV) vector genome, comprising the polynucleotide of the invention, wherein the AAV vector genome is capable of being packaged inside an AAV capsid.

Another aspect of the invention provides a recombinant adeno associated viral (rAAV) particle, comprising an AAV capsid, and an AAV vector genome comprising the polynucleotide of the invention, wherein the AAV vector genome is encapsidated within the AAV capsid.

In certain embodiments, the polynucleotide is operably linked to a transcriptional regulatory element. In certain embodiments, the transcriptional regulatory element comprises a promoter, such as a constitutive promoter, or a muscle-specific promoter.

Numerous muscle-specific promoters can be used to express the CpG reduced codon optimized polynucleotide of the invention, including, not limited to, CK8 promoter, cardiac troponin T (cTnT) promoter, CK7 promoter, CK9 promoter, truncated MCK (tMCK), myosin heavy chain (MHC) promoter, hybrid α-myosin heavy chain enhancer-/MCK enhancer-promoter (MHCK7), a muscle specific creatine kinase (MCK) promoter, human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

In certain embodiments, the muscle-specific promoter is a CK8 promoter.

In certain embodiments, the CK8 promoter comprises the nucleotide sequence of SEQ ID NO: 3.

In certain embodiments, the CK8 promoter is a modified CK8 promoter comprising an additional enhancer element. In certain embodiments, the modified CK8 promoter comprises SEQ ID NO: 6 (the basal CK8 promoter, a 269-bp fragment of the CK8 promoter of SEQ ID NO: 3), as well as one additional copy of a 130-bp enhancer (SEQ ID NO: 5) at the 5′ end. In certain embodiments, the modified CK8 promoter is CK8e promoter comprising the nucleotide sequence of SEQ ID NO: 4.

In certain embodiments, the vector genome further comprises a polyadenylation signal sequence.

In certain embodiments, the polyA signal sequence comprises SEQ ID NO: 8.

In certain embodiments, the polyA signal sequence comprises an SV40 polyadenylation signal sequence (e.g., SEQ ID NO: 9).

In certain embodiments, the polyA signal sequence comprises a bovine growth hormone (bGH) polyadenylation signal sequence (e.g., SEQ ID NO: 10).

In certain embodiments, the polyA signal sequence comprises a rabbit beta globin (rBG) polyadenylation signal sequence (e.g., SEQ ID NO: 11).

In certain embodiments, the vector genome further comprises a 3′ ITR sequence. The ITR sequence can be from any AAV, such as an AAV2 3′ ITR sequence.

In certain embodiments, the vector genome further comprises a 5′ ITR sequence. The ITR sequence can be from any AAV, such as an AAV2 5′ ITR sequence.

In certain embodiments, the vector genome further comprises a 5′ ITR sequence and a 3′ ITR sequence. The ITR sequences can be from any AAV, such as an AAV2 5′ and 3′ ITR sequences.

Inverted Terminal Repeat (ITR) sequences are important for initiation of viral DNA replication and circularization of adeno-associated virus genomes. Within the ITR sequences, secondary structures (e.g., stems and loops formed by palindromic sequences) are important one or more ITR functions in viral replication and/or packaging. Such sequence elements includes the RBE sequence (Rep binding element), RBE′ sequence, and the TRS (terminal resolution sequence).

In certain embodiments, the 5′ and/or 3′ ITR sequences are wild-type sequences.

In certain embodiments, the 5′ and/or 3′ ITR sequences are modified ITR sequences. For example, the most 5′ end or the most 3′ end of the wild-type ITR sequences may be deleted. The deletion can be up to 18, 17, 16, 15, 14, 13, 12, 11, 10, 9, 8, 7, 6, 5, 4, 3, 2, or 1 nucleotides.

In certain embodiments, up to 15 (such as exactly 15) nucleotides of the most 5′ end nucleotides, and/or up to 15 (such as exactly 15) nucleotides of the most 3′ end nucleotides, of the wild-type AAV2 ITR sequences may be deleted.

Thus the 5′ and/or 3′ modified ITR(s) may comprising up to 144, 143, 142, 141, 140, 139, 138, 137, 136, 135, 134, 133, 132, 131, 130, 129, 128, or 127-nt (such as 130 nucleotides) of the 145-nt wild-type AAV ITR sequences.

In certain embodiments, the modified ITR sequences comprise the RBE sequence, the RBE′ sequence, and/or the TRS of the wt ITR sequence.

In certain embodiments, the modified ITR sequences comprise both the RBE sequence and the RBE′ sequence.

In certain embodiments, the modified ITR sequences confer stability of the plasmids of the invention comprising the AAV vector genome (see below) in bacteria, such as stability during plasmid production.

In certain embodiments, the modified ITRs do not interfere with sequencing verification of the plasmids of the invention comprising the AAV vector genome.

In certain embodiments, the modified 5′ ITR sequence comprises a 5′ heterologous sequence that is not part of wild-type AAV 5′ ITR sequence. In certain embodiments, the modified 3′ ITR sequence comprises a 3′ heterologous sequence that is not part of wild-type AAV 3′ ITR sequence.

In certain embodiments, the modified 5′ ITR sequence comprises a 5′ heterologous sequence that is not part of wild-type AAV (e.g., wt AAV2) 5′ ITR sequence, and the modified 3′ ITR sequence comprises a 3′ heterologous sequence that is not part of wild-type AAV (e.g., wt AAV2) 3′ ITR sequence, wherein the 5′ heterologous sequence and the 3′ heterologous sequence are complementary to each other.

In certain embodiments, the 5′ heterologous sequence and the 3′ heterologous sequence each comprises a type II restriction endonuclease recognition sequence, such as recognition sequence for Sse8387I (CCTGCAGG), or recognition sequence for PacI (TTAATTAA).

In certain embodiments, the 5′ heterologous sequence comprises, consists essentially of, or consists of CCTGCAGGCAG (SEQ ID NO: 19), and the 3′ heterologous sequence comprises, consists essentially of, or consists of the reverse complement of SEQ ID NO: 19.

In certain embodiments, the 5′ heterologous sequence comprises, consists essentially of, or consists of TTAATTAAGG (SEQ ID NO: 22), and the 3′ heterologous sequence comprises, consists essentially of, or consists of the reverse complement of SEQ ID NO: 22.

In certain embodiments, the 5′ ITR and the 3′ ITR are both flip ITR's.

In certain embodiments, the 5′ ITR and the 3′ ITR are both flop ITR's.

In certain embodiments, the 5′ ITR and the 3′ ITR are independently flip or flop ITR's.

In certain embodiments, the 5′ ITR is a flip ITR, and the 3′ ITR is a flop ITR.

In certain embodiments, the 5′ ITR is a flop ITR, and the 3′ ITR is a flip ITR.

In certain embodiments, the 5′ ITR is a flip ITR, and the 3′ ITR is a flip ITR.

In certain embodiments, the 5′ ITR is a flop ITR, and the 3′ ITR is a flop ITR.

As used herein, a 5′ flip ITR has the B:B′ segment closer to the 5′-terminal than the C:C′ segment. A 3′ flip ITR has the B:B′ segment closer to the 3′-terminal than the C:C′ segment. A 5′ flop ITR has the C:C′ segment closer to the 5′-terminal than the B:B′ segment. A 3′ flop ITR has the C:C′ segment closer to the 3′-terminal than the B:B′ segment.

In certain embodiments, the modified 5′ ITR and the modified 3′ ITR are both flop ITRs, the modified 5′ ITR comprises a 5′ heterologous sequence that is not part of wild-type AAV2 5′ ITR sequence (such as SEQ ID NO: 19 or 22), and the modified 3′ ITR sequence comprises a 3′ heterologous sequence that is not part of wild-type AAV2 3′ ITR sequence, wherein the 5′ heterologous sequence and the 3′ heterologous sequence are complementary to each other, and each comprises a type II restriction endonuclease recognition sequence, such as recognition sequence for Sse8387I or PacI; optionally, said modified 5′ ITR sequence further comprises a deletion in the C:C′ segment, such as an 11-nts deletion AAAGCCCGGGC (SEQ ID NO: 23).

In certain embodiments, the 5′ ITR comprises, consists essentially of, or consists SEQ ID NO: 12.

(SEQ ID NO: 12)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT

In certain embodiments, the 5′ ITR comprises, consists essentially of, or consists SEQ ID NO: 24.

(SEQ ID NO: 24)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT

In certain embodiments, the 5′ ITR comprises, consists essentially of, or consists SEQ ID NO: 25.

(SEQ ID NO: 25)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT

In certain embodiments, the 5′ ITR comprises, consists essentially of, or consists SEQ ID NO: 26.

(SEQ ID NO: 26)
TTAATTAAGGCTGCGCGCTCGCTCGCTCACTGAGGCCGCCCGGGCAAAGC
CCGGGCGTCGGGCGACCTTTGGTCGCCCGGCCTCAGTGAGCGAGCGAGCG
CGCAGAGAGGGAGTGGCCAACTCCATCACTAGGGGTTCCT

In certain embodiments, the 3′ ITR comprises, consists essentially of, or consists SEQ ID NO: 13.

(SEQ ID NO: 13)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG

In certain embodiments, the 3′ ITR comprises, consists essentially of, or consists SEQ ID NO: 27.

(SEQ ID NO: 27)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGCTGCCTGCAGG

In certain embodiments, the 3′ ITR comprises, consists essentially of, or consists SEQ ID NO: 28.

(SEQ ID NO: 28)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGCCTTAATTAA

In certain embodiments, the 5′ ITR sequence is or comprises SEQ ID NO: 12, and the 3′ ITR sequence is or comprises SEQ ID NO: 13.

In certain embodiments, the 5′ ITR sequence is or comprises SEQ ID NO: 24, and the 3′ ITR sequence is or comprises SEQ ID NO: 27.

In certain embodiments, the 5′ ITR comprises up to 141 nts of the most 3′ nucleotides of the 145-nt wt AAV2 5′ ITR (e.g., a deletion of 4 or more most 5′ end of the 145-nt wt AAV2 5′ ITR).

In certain embodiments, the 5′ ITR comprises up to 130 nts of the most 3′ nucleotides of the 145-nt wt AAV2 5′ ITR (e.g., a deletion of 15 or more most 5′ end of the 145-nt wt AAV2 5′ ITR).

In certain embodiments, the 3′ ITR comprises up to 141 nts of the most 5′ nucleotides of the 145-nt wt AAV2 3′ ITR (e.g., a deletion of 4 or more most 3′ end of the 145-nt wt AAV2 3′ ITR).

In certain embodiments, the 3′ ITR comprises up to 130 nts of the most 5′ nucleotides of the 145-nt wt AAV2 3′ ITR (e.g., a deletion of 15 or more most 3′ end of the 145-nt wt AAV2 3′ ITR).

In certain embodiments, the 5′ and 3′ ITR sequences are compatible for AAV production in mammalian-cell based on triple transfection.

In certain embodiments, the 5′ and 3′ ITR sequences are compatible for AAV production in insect cell (e.g., Sf9) based on baculovirus vector (see below).

In certain embodiments, the 5′ and 3′ ITR sequences are compatible for AAV production in mammalian-cell based on HSV vectors (see below).

In certain embodiments, the vector genome further comprises an intron and/or an exon sequence that enhances expression of the microdystrophin. In certain embodiments, the intron/exon increases expression of the microdystrophin by up to 2-10 folds.

In certain embodiments, the intron comprises the sequence of a β-globin splice donor/IgG splice acceptor chimeric intron (see, for example, the chimeric intron in Promega pCMVTnT vector (Cat. No. L5620).

In certain embodiments, the intron comprises SEQ ID NO: 14.

(SEQ ID NO: 14)
gtatcaaggttacaagacaggtttaaggagaccaatagaaactgggctt
gtcgagacagagaagactcttgcgtttctgataggcacctattggtctt
actgacatccactttgcctttctctccacag

In certain embodiments, the promoter is CK8e promoter (infra) that comprises a 48 bp (SEQ ID NO: 7) or 50 bp (SEQ ID NO: 8) MCK UTR exon sequence that enhances expression.

In certain embodiments, the vector genome does not comprise intron and/or exon sequences that potentially enhances expression of the microdystrophin. Eliminating intron/exon sequences may improve packaging efficiency and increase packaging capacity for other sequence elements.

In certain embodiments, the vector genome further comprises a 5′ UTR sequence, and/or a 3′ UTR sequence.

In certain embodiments, the AAV vector genome or the rAAV viral particle of the invention comprises, consists essentially of, or consists of, from 5′ to 3′, the following sequence elements: (1) a 5′ ITR (such as a wild-type or modified AAV2 5′ ITR, e.g., the 145-nt wild-type AAV2 5′ ITR, or the 141-nt modified AAV2 5′ ITR (such as SEQ ID NO: 12)), (2) a muscle-specific promoter (such as a CK8 promoter (e.g., SEQ ID NO: 3) or a modified CK8 protein such as CK8e as described herein (SEQ ID NO: 4)); (3) any one of the CpG reduced/eliminated codon optimized polynucleotide of the invention (such as SEQ ID NO: 1 or a nucleotide sequence at least 70%, 75%, 80%, 85%, 90%, 95%, 96%, 97%, 98%, 99%, 99.2%, 99.4%, 99.6%, 99.8%, or 99.9% identical thereto); (4) a polyA signal sequence (such as any one of SEQ ID NOs: 8-11); and (5) a 3′ ITR (such as a wild-type or modified AAV2 3′ ITR, e.g., the 145-nt wild-type AAV2 3′ ITR, or the 141-nt modified AAV2 3′ ITR (Such as SEQ ID NO: 13)); or a nucleotide sequence at least 90%, 95%, 96%, 97%, 98%, 99%, 99.5%, 99.7% or 99.9% identical to the AAV vector genome. Optionally, immediately before (3), there is a KOZAK sequence comprising ACC immediately 5′ to the ATG start codon.

In certain embodiments, the AAV vector genome or the rAAV viral particle of the invention comprises, consists essentially of, or consists of, from 5′ to 3′, the following sequence elements: (1) a 5′ ITR (such as SEQ ID NO: 12), (2) a CK8 promoter (e.g., SEQ ID NO: 3); (3) any one of the CpG reduced/eliminated codon optimized polynucleotide of the invention (such as SEQ ID NO: 1); (4) a polyA signal sequence (such as SEQ ID NO: 8); and (5) a 3′ ITR (such as SEQ ID NO: 13); or a nucleotide sequence at least 90%, 95%, 96%, 97%, 98%, 99%, 99.5%, 99.7% or 99.9% identical to the AAV vector genome. Optionally, immediately before (3), there is a KOZAK sequence comprising ACC immediately 5′ to the ATG start codon.

In certain embodiments, the AAV vector genome or the rAAV viral particle of the invention comprises, consists essentially of, or consists of the nucleotide sequence of SEQ ID NO: 15, or a nucleotide sequence at least 90%, 95%, 96%, 97%, 98%, 99%, 99.5%, 99.7% or 99.9% identical thereto.

In certain embodiments, the rAAV viral particle of the invention comprise a capsid of the serotype of SLB-101, AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, or AAVhu37.

In certain embodiments, the serotype is SLB-101 (e.g., the VP1 capsid sequence is SEQ ID NO: 21) or AAV9 (e.g., the VP1 capsid sequence is SEQ ID NO: 20).

Another aspect of the invention provides a recombinant adeno-associated virus (rAAV) viral particle, comprising an SLB-101 or AAV9 capsid, and a vector genome encapsidated therein, wherein said vector genome comprises a polynucleotide sequence encoding the MD5 microdystrophin of SEQ ID NO: 2.

In certain embodiments, the polynucleotide sequence comprises the nucleotide sequence of SEQ ID NO: 1, or a nucleotide sequence at least 70%, 75%, 80%, 85%, 90%, 95%, 96%, 97%, 98%, 99%, 99.2%, 99.4%, 99.6%, 99.8%, or 99.9% identical thereto.

In certain embodiments, the polynucleotide sequence comprises a nucleotide sequence at least 70%, 75%, 80%, 85%, 90%, 95%, 96%, 97%, 98%, 99% identical to SEQ ID NO: 1, and is identical to SEQ ID NO: 1 at each capitalized nucleotides or differ by no more than 1, 2, 3, 4, 5, 6, 7, 8, 9, 10, 11, 12, 13, 14, 15, 16, 17, 18, 19, or 20 capitalized nucleotides.

In certain embodiments, the polynucleotide sequence substantially lacks CpG islands (e.g., no more than 1, 2, 3, 4, 5, 6, 7, 8, 9, or 10 CpG islands).

In certain embodiments, the vector genome comprises a muscle-specific control element operably linked to the polynucleotide sequence.

In certain embodiments, the muscle-specific control element comprises a CK8 promoter, such as the CK8 promoter of the nucleotide sequence of SEQ ID NO: 3 or 4.

In certain embodiments, the vector genome further comprises a polyadenylation signal sequence, such as any one of SEQ ID NOs: 8-11.

In certain embodiments, the vector genome further comprises a 3′ ITR sequence, such as SEQ ID NO: 13; and a 5′ ITR sequence, such as SEQ ID NO: 12.

Another aspect of the invention provides a pharmaceutical composition comprising the polynucleotide of the invention, the rAAV vector genome or the rAAV viral particle of the invention, and a pharmaceutically acceptable carrier.

In certain embodiments, the pharmaceutical composition is suitable or formulated for intravenous, subcutaneous, intramuscular, intradermal, intraperitoneal, or intrathecal administration.

Another aspect of the invention provides a method of treating a muscular dystrophy in a human in need thereof, the method comprising administering to the human a therapeutically effective amount of the polynucleotide of the invention, the rAAV vector genome or the rAAV viral particle of the invention, or the pharmaceutical composition of the invention.

In certain embodiments, the muscular dystrophy is characterized by a loss-of-function a mutation in the dystrophin gene.

In certain embodiments, the muscular dystrophy is Duchenne muscular dystrophy (DMD), Becker muscular dystrophy (BMD), or X-linked dilated cardiomyopathy.

In certain embodiments, the rAAV viral particle is administered at a dose of about 1×10¹² to about 1×10¹⁶ vector genome (vg)/kg, or about 1×10¹³ to about 1×10¹⁵ vector genome (vg)/kg.

Another aspect of the invention provides a host cell comprising the polynucleotide of the invention, or the rAAV vector genome or the rAAV viral particle of the invention.

In certain embodiments, the host cell is a HeLa cell, a Cos7 cell, a HEK293 cell, an A549 cell, a BHK cell, a Vero cell, an RD cell, an HT-1080 cell, an ARPE-19 cell, or a MRC-5 cell.

In certain embodiments, the host cell is a HeLa cell or a 293/293T cell.

With the inventions generally described above, the following sections provide more detailed description for specific aspects of the invention. Thus any one embodiment described herein, including those described only in the examples or the claims, can be combined with any one or more additional embodiments of the invention unless expressly disclaimed or improper.

### 2. CpG Condon Optimized Polynucleotide Encoding Microdystrophin, AAV Vector Genome

In one aspect, the invention described herein provides a codon optimized polynucleotide sequence, such as SEQ ID NO: 1, which encodes a microdystrophin protein of SEQ ID NO: 2, and which has reduced number of CpG sites/islands, or has substantially eliminated CpG islands. The polynucleotide sequence of SEQ ID NO: 1, and the protein sequence of SEQ ID NO: 2, are provided below.

(SEQ ID NO: 1)
atgctgtggtgggaggaagtggaagattgctacgagcgcgaggacgtgcagaagaaaaccttcaccaa
atgggtcaacgcccagttcagcaagttcggcaagcagcacatcgagaacctgttcagcgacctgcagg
acggcagacggctgctggatctgctggaaggcctgaccggacagaagctgcccaaagagaagggcagc
accagagtgcacgccctgaacaacgtgaacaaggccctgcgggtgctgcagaacaacaaTgtggacct
GgtgaacatTggcagcacAgacatTgtggaTggcaaccacaagctgaccctgggcctgatctggaaca
tcatcctgcactggcaagtgaagaacgtgatgaagaacatcatggccggcctgcagcagaccaacagc
gagaagatcctgctgagctgggtgcgccagagcaccagaaactacccccaagtgaacgtgatcaactt
caccacctcttggagcgacggcctggccctgaatgccctgatccacagccacagacccgacctgttIg
actggaacagTgtGgtgtgtcagcagagcgccacccagaggctggaacacgccttcaatatcgccaga
taccagctgggcatTgagaagctgctggaccccgaggatgtggacaccacctaccccgacaagaaatc
catcctgatgtatatcaccagcctgttccaggtgctgcctcagcaggtgtccatcgaggccatccagg
aagtggaaatgctgcccagaccccccaaagtgaccaaagaggaacacttccagctgcaccaccagatg
cactactctcagcagatcaccgtgtccctggcccagggctacgagagaaccagcagccccaagccccg
gttcaagagctacgcctatacccaggccgcctacgtgaccaccagcgaccctaccagaagcccattcc
ccagccagcatctggaagcccccgaggacaagagcttcggcagcagcctgatggaaagcgaagtgaac
ctggatagataccagaccgccctggaagaggtgctgtcctggctgctgagcgccgaggatacactgca
ggctcagggcgagatcagcaaTgaTgtggaagtGgtgaaggaccagttccacacccacgagggctaca
tgatggacctgacagcccaccagggcagagtgggcaacattctgcagctgggctccaagctgatcggc
accggcaagctgagcgaggacgaagagacagaggtgcaggaacagatgaacctgctgaacagcagatg
ggagtgcctgagagtggccagcatggaaaagcagagcaacctgcacagctacgtgcccagcacctacc
tgaccgagatcacccatgtgtcccaggccctgctggaagtggaacagctgctgaacgcccccgatctg
tgcgccaaggacttcgaggatctgttcaagcaggaagagagcctgaagaatatcaaggactctctgca
gcagtccagcggcagaatcgacatcatccacagcaagaaaacagccgccctgcagtccgccacccccg
tggaaagagtgaagctgcaggaagccctgtcccagctggacttccagtgggagaaagtgaacaagatg
tacaaggaccggcagggcagattTgaccgcagTgtggaaaagtggAggAggttccactacgacatcaa
gatcttcaaccagtggctgacAgaggccgagcagttcctgagaaagacccagatccccgagaactggg
agcacgccaagtacaagtggtatctgaaagaactgcaggatggcatTggccagagacagacAgtGgtg
cggacactgaatgccaccggcgaggaaatcatccagcagagcagcaagaccgacgccagtattctgca
ggaaaagctgggcagcctgaacctgagatggcaggaagtgtgcaagcagctgtccgaccggaagaaga
gactggaagaacagagTgaccagtggaagcggctgcatctgtcactgcaggaactgctGgtgtggctg
cagctgaaggaTgaTgagctgagcagacaggcccctatTggcggcgattttcccgcAgtgcagaaaca
gaacgaTgtgcaccgggccttcaagagagagctgaaaacaaaagaaccAgtgatcatgagcaccctgg
aaacAgtgcggatctttctgaccgagcagcccctggaaggactggaaaaactgtaccaggaacccaga
gagctgccccctgaagaacgggcccagaacgtgaccagactgctgAggaagcaggccgaggaagtgaa
cacAgaatgggagaagctgaacctgcactcTgcTgactggcagAggaagatTgaTgagacactggaac
ggctgcaggaactgcaggaggccacAgacgagctggacctgaaactgagacaggccgaagtgatcaag
ggcagctggcagccagtgggcgacctgctgatcgacagcctgcaggatcacctggaaaaagtgaaagc
cctgagaggcgagatTgcccccctgaaagaaaaTgtgtcccaTgtgaacgacctggcccggcagctga
caacactgggcatccagctgagcccctacaacctgtccacactggaagatctgaacacccggtggaaa
ctgctgcaggtggccgtggaagatagagtgcggcagctgcacgaggcccacagagattttggccctgc
ctcccagcacttcctgagcacatctgtgcagggcccctgggagagagccatctcccccaacaaggtgc
cctactacatcaaccacgagacacagaccacctgttgggaccaccccaagatgacAgagctgtaccag
agcctggccgacctgaacaatgtgAggttcagTgcctacAggaccgccatgaagctgcggagactgca
gaaagctctgtgcctggacctgctgtccctgtccgccgcttgtgatgccctggaccagcacaacctga
agcagaacgaccagcccatggatatcctgcagatcatcaactgcctgaccaccatctacgaccgcctg
gaacaggaacacaacaacctGgtgaatgtgcccctgtgIgtggacatgtgcctgaattggctgctgaa
tgtgtacgacaccggccggacaggccggatcagagtgctgagcttcaagaccggcatcatcagcctgt
gcaaggcccacctggaagataagtaccgctacctgttcaaacaggtggccagctccaccggcttttgc
gaccagagaaggctgggcctgctgctgcacgacagcatccagatccctagacagctgggcgaggtggc
ctctttTggcggcagcaatatTgagcctagTgtgcggagctgcttccagttTgccaacaacaagcccg
agatTgaggccgccctgttcctggactggatgcggctggaaccccagagcatggtgtggctgcctgtg
ctgcatagagtggccgctgccgagacagccaagcaccaggccaagtgcaacatctgcaaagagtgccc
catcatcggcttccggtacagaagcctgaagcacttcaactacgatatctgccagagctgctttttca
gcggacgggtggccaagggccacaaaatgcactaccccatggtggaatactgcacccccaccacctcc
ggggaggatgtgcgggattttgccaaggtgctgaaaaacaagttccggaccaagcgctacttTgccaa
acacccccggatgggctatctgcccgtgcagacagtgctggaaggcgacaacatggaaaccgacacca
tgtag
(SEQ ID NO: 2)
MLWWEEVEDCYEREDVQKKTFTKWVNAQFSKFGKQHIENLFSDLQDGRRLLDLLEGLTGQKLPKEKGS
TRVHALNNVNKALRVLQNNNVDLVNIGSTDIVDGNHKLTLGLIWNIILHWQVKNVMKNIMAGLQQTNS
EKILLSWVRQSTRNYPQVNVINFTTSWSDGLALNALIHSHRPDLFDWNSVVCQQSATQRLEHAFNIAR
YQLGIEKLLDPEDVDTTYPDKKSILMYITSLFQVLPQQVSIEAIQEVEMLPRPPKVTKEEHFQLHHQM
HYSQQITVSLAQGYERTSSPKPRFKSYAYTQAAYVTTSDPTRSPFPSQHLEAPEDKSFGSSLMESEVN
LDRYQTALEEVLSWLLSAEDTLQAQGEISNDVEVVKDQFHTHEGYMMDLTAHQGRVGNILQLGSKLIG
TGKLSEDEETEVQEQMNLLNSRWECLRVASMEKQSNLHSYVPSTYLTEITHVSQALLEVEQLLNAPDL
CAKDFEDLFKQEESLKNIKDSLQQSSGRIDIIHSKKTAALQSATPVERVKLQEALSQLDFQWEKVNKM
YKDRQGRFDRSVEKWRRFHYDIKIFNQWLTEAEQFLRKTQIPENWEHAKYKWYLKELQDGIGQRQTVV
RTLNATGEEIIQQSSKTDASILQEKLGSLNLRWQEVCKQLSDRKKRLEEQSDQWKRLHLSLQELLVWL
QLKDDELSRQAPIGGDFPAVQKQNDVHRAFKRELKTKEPVIMSTLETVRIFLTEQPLEGLEKLYQEPR
ELPPEERAQNVTRLLRKQAEEVNTEWEKLNLHSADWQRKIDETLERLQELQEATDELDLKLRQAEVIK
GSWQPVGDLLIDSLQDHLEKVKALRGEIAPLKENVSHVNDLARQLTTLGIQLSPYNLSTLEDLNTRWK
LLQVAVEDRVRQLHEAHRDFGPASQHFLSTSVQGPWERAISPNKVPYYINHETQTTCWDHPKMTELYQ
SLADLNNVRFSAYRTAMKLRRLQKALCLDLLSLSAACDALDQHNLKQNDQPMDILQIINCLTTIYDRL
EQEHNNLVNVPLCVDMCLNWLLNVYDTGRIGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVWLPV
LHRVAAAETAKHQAKCNICKECPIIGFRYRSLKHFNYDICQSCFFSGRVAKGHKMHYPMVEYCTPTTS
GEDVRDFAKVLKNKFRTKRYFAKHPRMGYLPVQTVLEGDNMETDTM

As used herein, “codon-optimized” polynucleotide coding sequence refers to a polynucleotide sequence that has been altered/changed in some respect, such that the resulting codons are optimal for expression in a particular cell, host, or system, such as in a specific mammalian (human) cell type, e.g., muscle cells. Codon optimization does not alter the amino acid sequence of the encoded protein, i.e., the codon optimized polynucleotide coding sequence, and the native sequence based on which codon optimization was performed, encode the same amino acid sequence.

The polynucleotides of the invention, such as SEQ ID NO: 1, encode a microdystrophin protein known as “microD5,” “MD5,” or “pD5” (see SEQ ID NO: 2). The micro-dystrophin protein provides stability to the muscle membrane during muscle contraction, e.g., micro-dystrophin acts as a shock absorber during muscle contraction. MD5 is a specific engineered 5-repeat microdystrophin protein that contains, from N- to C-terminus, the N-terminal actin binding domain, Hinge region 1 (H1), spectrin-like repeats R1, R16, R17, R23, and R24, Hinge region 4 (H4), and the C-terminal dystroglycan binding domain of the human full-length dystrophin protein. The protein sequence of this 5-repeat microdystrophin and the related dystrophin minigene are described in U.S. Pat. No. 10,479,821 & WO2016/115543 (incorporated herein by reference).

In SEQ ID NO: 1 shown above, certain nucleotides are marked up as capital letters, and these nucleotides are collectively referred to herein as “capitalized nucleotides of SEQ ID NO: 1.” Specifically, the capitalized nucleotides of SEQ ID NO: 1 includes nucleotides 264, 273, 282, 291, 297, 303, 543, 555, 558, 627, 1110, 1113, 1122, 1656, 1665, 1678, 1681, 1722, 1815, 1830, 1833, 1989, 2031, 2052, 2055, 2079, 2097, 2115, 2157, 2181, 2290, 2316, 2343, 2346, 2356, 2364, 2367, 2406, 2532, 2550, 2559, 2844, 2881, 2889, 2896, 3081, 3099, 3339, 3354, 3363, 3384, 3405, and 3735 of SEQ ID NO: 1.

In certain embodiments, the polynucleotides of the invention not only encode the same protein (i.e., SEQ ID NO: 2), but also share the same set of capitalized nucleotides of SEQ ID NO: 1, yet they differ from SEQ ID NO: 1 at nucleotide positions other than the capitalized nucleotides of SEQ ID NO: 1.

In certain embodiments, the polynucleotides of the invention not only encode the same protein (i.e., SEQ ID NO: 2), but are also substantially identical to SEQ ID NO: 1 at the capitalized nucleotides of SEQ ID NO: 1, despite additional sequence changes (e.g., to result in 70%, 75%, 80%, 85%, 90%, 95%, 96%, 97%, 98%, 99%, 99.2%, 99.4%, 99.6%, 99.8%, or 99.9% overall sequence identity) in positions of SEQ ID NO: 1 other than the capitalized nucleotides. In certain embodiments, the polynucleotides of the invention is identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides.

In certain embodiments, the polynucleotide of the invention comprises, consists essentially of, or consists of a nucleotide sequence at least 95% identical to SEQ ID NO: 1. That is, the polynucleotide of the invention encodes the microdystrophin of SEQ ID NO: 2, and further, the polynucleotide of the invention is (1) identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides; and/or (2) substantially lacks CpG islands (e.g., no more than 1, 2, 3, 4, 5, 6, 7, 8, 9, or 10 CpG islands based on EMBOSS Cpgplot analysis).

In certain embodiments, the polynucleotide of the invention comprises, consists essentially of, or consists of a nucleotide sequence at least 97% identical to SEQ ID NO: 1. That is, the polynucleotide of the invention encodes the microdystrophin of SEQ ID NO: 2, and further, the polynucleotide of the invention is (1) identical to SEQ ID NO: 1 at each capitalized nucleotide, or differ by no more than 1, 2, 3, 4, 5, 6, 7, 8, 9, 10, 11, 12, 13, 14, 15, 16, 17, 18, 19, or 20 capitalized nucleotides; and/or (2) substantially lacks CpG islands (e.g., no more than 1, 2, 3, 4, 5, 6, 7, 8, 9, or 10 CpG islands based on EMBOSS Cpgplot analysis).

In certain embodiments, the polynucleotide of the invention comprises, consists essentially of, or consists of a nucleotide sequence at least 99% identical to SEQ ID NO: 1. That is, the polynucleotide of the invention encodes the microdystrophin of SEQ ID NO: 2, and further, the polynucleotide of the invention is (1) identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides; and/or (2) substantially lacks CpG islands (e.g., no more than 1, 2, 3, 4, 5, 6, 7, 8, 9, or 10 CpG islands based on EMBOSS Cpgplot analysis).

Sequence percentage identity between any two or more related or unrelated polynucleotides, or between any two or more related or unrelated protein sequences, can be aligned and the percentage of the matches between the nucleotides or amino acid residues, respectively, can be calculated using any art recognized methods, such as the NCBI Basic Local Alignment Search Tool (BLAST) (Altschul et al., J. Mol. Biol. 215:403-10, 1990), which is available from online sources, such as the National Center for Biological Information (NCBI) website, for use in connection with the sequence analysis programs blastp, blastn, blastx, tblastn and tblastx, depending on the type of query and database. Similar web-based tools can be found at the EMBL-EBI website.

In certain embodiments, the polynucleotide of the invention substantially lacks CpG islands (e.g., no more than 1, 2, 3, 4, 5, 6, 7, 8, 9, or 10 CpG islands based on EMBOSS Cpgplot analysis).

In certain embodiments, the polynucleotide of the invention substantially fails to induce TLR9 activation, such as in an in vitro assay as described in Example 2.

In certain embodiments, the polynucleotide of the invention comprises, consists essentially of, or consists of the nucleotide sequence of SEQ ID NO: 1.

Another aspect of the invention, provides an adeno associated virus (AAV) vector genome, comprising any polynucleotide of the invention, wherein the AAV vector genome is capable of being packaged inside an AAV capsid.

The packaging capacity of a typical AAV is generally about 4.7 kb, including about 0.2-0.3 kb of 5′ and 3′ ITR sequences, at least one (maybe both) of which are structural elements required for AAV vector genome packaging into the capsid.

In certain embodiments, the AAV vector genome comprises certain ITR structural element, such as the Rep binding element (RBE), the internal hairpin within the TR (RBE′), and the terminal resolution site (TRS).

Another aspect of the invention, provides a recombinant adeno associated viral (rAAV) particle, comprising an AAV capsid, and an AAV vector genome comprising any polynucleotide of the invention, wherein the AAV vector genome is encapsidated within the AAV capsid.

In certain embodiments, the (CpG codon optimized) polynucleotide is operably linked to a transcriptional regulatory element. In certain embodiments, the transcriptional regulatory element comprises a promoter, such as a constitutive promoter, or a tissue-specific promoter (e.g., muscle specific promoter) (infra). An exemplary promoter is CK8 or variant thereof (infra).

In certain embodiments, the vector genome further comprises a polyadenylation signal sequence, such as the polyA signal sequence of any one of SEQ ID NOs: 8-11 (infra).

In certain embodiments, the vector genome further comprises a 3′ ITR sequence, such as an AAV2 3′ ITR sequence. In certain embodiments, the vector genome further comprises a 5′ ITR sequence, such as an AAV2 5′ ITR sequence. In certain embodiments, the 5′ ITR sequence, and/or the 3′ ITR sequence comprise or are SEQ ID NOs: 12 and 13, respectively.

In certain embodiments, the vector genome further comprises an intron and/or an exon sequence that enhances expression of the microdystrophin. In certain embodiments, the vector genome does not comprise intron and/or exon sequence that otherwise enhances expression of the microdystrophin.

In certain embodiments, the vector genome further comprises a 5′ UTR sequence, and/or a 3′ UTR sequence.

In certain embodiments, the AAV vector genome or the rAAV viral particle of the invention comprises, consists essentially of, or consists of, from 5′ to 3′, the following sequence elements: (1) a 5′ ITR (such as a wild-type or modified AAV2 5′ ITR, e.g., the 145-nt wild-type AAV2 5′ ITR, or the 141-nt modified AAV2 5′ ITR), (2) a muscle-specific promoter (such as a CK8 promoter or a modified CK8 protein such as CK8e as described herein); (3) any one of the CpG reduced/eliminated codon optimized polynucleotide of the invention (such as SEQ ID NO: 1 or a nucleotide sequence at least 70%, 75%, 80%, 85%, 90%, 95%, 96%, 97%, 98%, 99%, 99.2%, 99.4%, 99.6%, 99.8%, or 99.9% identical thereto); (4) a polyA signal sequence (such as any one of SEQ ID NOs: 8-11); and (5) a 3′ ITR (such as a wild-type or modified AAV2 3′ ITR, e.g., the 145-nt wild-type AAV2 3′ ITR, or the 141-nt modified AAV2 3′ ITR).

In certain embodiments, the AAV vector genome or the rAAV viral particle of the invention comprises, consists essentially of, or consists of the nucleotide sequence of SEQ ID NO: 15, or a nucleotide sequence at least 90%, 95%, 96%, 97%, 98%, 99%, 99.5%, 99.7% or 99.9% identical thereto.

(SEQ ID NO: 15)
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTGCGGCCGGCGCGCCACTTTAGACTAGCATGCTGCCCATGTAAGGAGGCAAGGCCTGGGGACAC
CCGAGATGCCTGGTTATAATTAACCCAGACATGTGGCTGCCCCCCCCCCCCCAACACCTGCTGCCTCT
AAAAATAACCCTGCATGCCATGTTCCCGGCGAAGGGCCAGCTGTCCCCCGCCAGCTAGACTCAGCACT
TAGTTTAGGAACCAGTGAGCAAGTCAGCCCTTGGGGCAGCCCATACAAGGCCATGGGGCTGGGCAAGC
TGCACGCCTGGGTCCGGGGTGGGCACGGTGCCCGGGCAACGAGCTGAAAGCTCATCTGCTCTCAGGGG
CCCCTCCCTGGGGACAGCCCCTCCTGGCTAGTCACACCCTGTAGGCTCCTCTATATAACCCAGGGGCA
CAGGGGCTGCCCTCATTCTACCACCACCTCCACAGCACAGACAGACACTCAGGAGCCAGCCAAAACTA
GAACCATGCTGTGGTGGGAGGAAGTGGAAGATTGCTACGAGCGCGAGGACGTGCAGAAGAAAACCTTC
ACCAAATGGGTCAACGCCCAGTTCAGCAAGTTCGGCAAGCAGCACATCGAGAACCTGTTCAGCGACCT
GCAGGACGGCAGACGGCTGCTGGATCTGCTGGAAGGCCTGACCGGACAGAAGCTGCCCAAAGAGAAGG
GCAGCACCAGAGTGCACGCCCTGAACAACGTGAACAAGGCCCTGCGGGTGCTGCAGAACAACAATGTG
GACCTGGTGAACATTGGCAGCACAGACATTGTGGATGGCAACCACAAGCTGACCCTGGGCCTGATCTG
GAACATCATCCTGCACTGGCAAGTGAAGAACGTGATGAAGAACATCATGGCCGGCCTGCAGCAGACCA
ACAGCGAGAAGATCCTGCTGAGCTGGGTGCGCCAGAGCACCAGAAACTACCCCCAAGTGAACGTGATC
AACTTCACCACCTCTTGGAGCGACGGCCTGGCCCTGAATGCCCTGATCCACAGCCACAGACCCGACCT
GTTTGACTGGAACAGTGTGGTGTGTCAGCAGAGCGCCACCCAGAGGCTGGAACACGCCTTCAATATCG
CCAGATACCAGCTGGGCATTGAGAAGCTGCTGGACCCCGAGGATGTGGACACCACCTACCCCGACAAG
AAATCCATCCTGATGTATATCACCAGCCTGTTCCAGGTGCTGCCTCAGCAGGTGTCCATCGAGGCCAT
CCAGGAAGTGGAAATGCTGCCCAGACCCCCCAAAGTGACCAAAGAGGAACACTTCCAGCTGCACCACC
AGATGCACTACTCTCAGCAGATCACCGTGTCCCTGGCCCAGGGCTACGAGAGAACCAGCAGCCCCAAG
CCCCGGTTCAAGAGCTACGCCTATACCCAGGCCGCCTACGTGACCACCAGCGACCCTACCAGAAGCCC
ATTCCCCAGCCAGCATCTGGAAGCCCCCGAGGACAAGAGCTTCGGCAGCAGCCTGATGGAAAGCGAAG
TGAACCTGGATAGATACCAGACCGCCCTGGAAGAGGTGCTGTCCTGGCTGCTGAGCGCCGAGGATACA
CTGCAGGCTCAGGGCGAGATCAGCAATGATGTGGAAGTGGTGAAGGACCAGTTCCACACCCACGAGGG
CTACATGATGGACCTGACAGCCCACCAGGGCAGAGTGGGCAACATTCTGCAGCTGGGCTCCAAGCTGA
TCGGCACCGGCAAGCTGAGCGAGGACGAAGAGACAGAGGTGCAGGAACAGATGAACCTGCTGAACAGC
AGATGGGAGTGCCTGAGAGTGGCCAGCATGGAAAAGCAGAGCAACCTGCACAGCTACGTGCCCAGCAC
CTACCTGACCGAGATCACCCATGTGTCCCAGGCCCTGCTGGAAGTGGAACAGCTGCTGAACGCCCCCG
ATCTGTGCGCCAAGGACTTCGAGGATCTGTTCAAGCAGGAAGAGAGCCTGAAGAATATCAAGGACTCT
CTGCAGCAGTCCAGCGGCAGAATCGACATCATCCACAGCAAGAAAACAGCCGCCCTGCAGTCCGCCAC
CCCCGTGGAAAGAGTGAAGCTGCAGGAAGCCCTGTCCCAGCTGGACTTCCAGTGGGAGAAAGTGAACA
AGATGTACAAGGACCGGCAGGGCAGATTTGACCGCAGTGTGGAAAAGTGGAGGAGGTTCCACTACGAC
ATCAAGATCTTCAACCAGTGGCTGACAGAGGCCGAGCAGTTCCTGAGAAAGACCCAGATCCCCGAGAA
CTGGGAGCACGCCAAGTACAAGTGGTATCTGAAAGAACTGCAGGATGGCATTGGCCAGAGACAGACAG
TGGTGCGGACACTGAATGCCACCGGCGAGGAAATCATCCAGCAGAGCAGCAAGACCGACGCCAGTATT
CTGCAGGAAAAGCTGGGCAGCCTGAACCTGAGATGGCAGGAAGTGTGCAAGCAGCTGTCCGACCGGAA
GAAGAGACTGGAAGAACAGAGTGACCAGTGGAAGCGGCTGCATCTGTCACTGCAGGAACTGCTGGTGT
GGCTGCAGCTGAAGGATGATGAGCTGAGCAGACAGGCCCCTATTGGCGGCGATTTTCCCGCAGTGCAG
AAACAGAACGATGTGCACCGGGCCTTCAAGAGAGAGCTGAAAACAAAAGAACCAGTGATCATGAGCAC
CCTGGAAACAGTGCGGATCTTTCTGACCGAGCAGCCCCTGGAAGGACTGGAAAAACTGTACCAGGAAC
CCAGAGAGCTGCCCCCTGAAGAACGGGCCCAGAACGTGACCAGACTGCTGAGGAAGCAGGCCGAGGAA
GTGAACACAGAATGGGAGAAGCTGAACCTGCACTCTGCTGACTGGCAGAGGAAGATTGATGAGACACT
GGAACGGCTGCAGGAACTGCAGGAGGCCACAGACGAGCTGGACCTGAAACTGAGACAGGCCGAAGTGA
TCAAGGGCAGCTGGCAGCCAGTGGGCGACCTGCTGATCGACAGCCTGCAGGATCACCTGGAAAAAGTG
AAAGCCCTGAGAGGCGAGATTGCCCCCCTGAAAGAAAATGTGTCCCATGTGAACGACCTGGCCCGGCA
GCTGACAACACTGGGCATCCAGCTGAGCCCCTACAACCTGTCCACACTGGAAGATCTGAACACCCGGT
GGAAACTGCTGCAGGTGGCCGTGGAAGATAGAGTGCGGCAGCTGCACGAGGCCCACAGAGATTTTGGC
CCTGCCTCCCAGCACTTCCTGAGCACATCTGTGCAGGGCCCCTGGGAGAGAGCCATCTCCCCCAACAA
GGTGCCCTACTACATCAACCACGAGACACAGACCACCTGTTGGGACCACCCCAAGATGACAGAGCTGT
ACCAGAGCCTGGCCGACCTGAACAATGTGAGGTTCAGTGCCTACAGGACCGCCATGAAGCTGCGGAGA
CTGCAGAAAGCTCTGTGCCTGGACCTGCTGTCCCTGTCCGCCGCTTGTGATGCCCTGGACCAGCACAA
CCTGAAGCAGAACGACCAGCCCATGGATATCCTGCAGATCATCAACTGCCTGACCACCATCTACGACC
GCCTGGAACAGGAACACAACAACCTGGTGAATGTGCCCCTGTGTGTGGACATGTGCCTGAATTGGCTG
CTGAATGTGTACGACACCGGCCGGACAGGCCGGATCAGAGTGCTGAGCTTCAAGACCGGCATCATCAG
CCTGTGCAAGGCCCACCTGGAAGATAAGTACCGCTACCTGTTCAAACAGGTGGCCAGCTCCACCGGCT
TTTGCGACCAGAGAAGGCTGGGCCTGCTGCTGCACGACAGCATCCAGATCCCTAGACAGCTGGGCGAG
GTGGCCTCTTTTGGCGGCAGCAATATTGAGCCTAGTGTGCGGAGCTGCTTCCAGTTTGCCAACAACAA
GCCCGAGATTGAGGCCGCCCTGTTCCTGGACTGGATGCGGCTGGAACCCCAGAGCATGGTGTGGCTGC
CTGTGCTGCATAGAGTGGCCGCTGCCGAGACAGCCAAGCACCAGGCCAAGTGCAACATCTGCAAAGAG
TGCCCCATCATCGGCTTCCGGTACAGAAGCCTGAAGCACTTCAACTACGATATCTGCCAGAGCTGCTT
TTTCAGCGGACGGGTGGCCAAGGGCCACAAAATGCACTACCCCATGGTGGAATACTGCACCCCCACCA
CCTCCGGGGAGGATGTGCGGGATTTTGCCAAGGTGCTGAAAAACAAGTTCCGGACCAAGCGCTACTTT
GCCAAACACCCCCGGATGGGCTATCTGCCCGTGCAGACAGTGCTGGAAGGCGACAACATGGAAACCGA
CACCATGTAGGAAGTCTTTTAATAAAAGATCCTTATTTTCATTGGATCTGTGTGTTGGTTTTTTGTGT
CAGCGGCCGCAGGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGAGCGAGCGAGCGC
GCAGCTGCCTGCAGG

### 3. Promoters

In certain embodiments, the codon optimized microdystrophin coding sequence is operably linked to a transcriptional regulatory element that includes a promoter operably linked to and is capable of driving the transcription of the microdystrophin coding sequence of the invention. The transcriptional regulatory element may further comprise one or more introns or exons that enhance expression of the microdystrophin encoded by the CpG reduced polynucleotide of the invention.

In certain embodiments, the transcriptional regulatory element comprises a constitutive promoter, such as a CMV promoter, a CAG promoter, an EF-1α promoter, a CB promoter, or a derivative thereof.

In certain embodiments, the transcriptional regulatory element comprises a muscle-specific control element.

For example, the muscle-specific control element can be: CK8 promoter, cardiac troponin T (cTnT) promoter, CK7 promoter, CK9 promoter, truncated MCK (tMCK), myosin heavy chain (MHC) promoter, hybrid α-myosin heavy chain enhancer-/MCK enhancer-promoter (MHCK7), a muscle specific creatine kinase (MCK) promoter, human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

In certain embodiments, muscle-specific control element is 5′ to a heterologous intron sequence (that enhanced microdystrophin expression), which is 5′ to the microdystrophin coding sequence of the invention, which is 5′ to an optional 3′-UTR region including a translation stop codon (such as TAG), a polyA adenylation signal (such as AATAAA), and an mRNA cleavage site (such as CA).

In certain embodiments, the muscle-specific control element comprises a CK8 promoter, such as one with the following sequence:

(CK8 PROMOTER, SEQ ID NO: 3)
TAGACTAGCATGCTGCCCATGTAAGGAGGCAAGGCCTGGGGACACCCGA
GATGCCTGGTTATAATTAACCCAGACATGTGGCTGCCCCCCCCCCCCCA
ACACCTGCTGCCTCTAAAAATAACCCTGCATGCCATGTTCCCGGCGAAG
GGCCAGCTGTCCCCCGCCAGCTAGACTCAGCACTTAGTTTAGGAACCAG
TGAGCAAGTCAGCCCTTGGGGCAGCCCATACAAGGCCATGGGGCTGGGC
AAGCTGCACGCCTGGGTCCGGGGTGGGCACGGTGCCCGGGCAACGAGCT
GAAAGCTCATCTGCTCTCAGGGGCCCCTCCCTGGGGACAGCCCCTCCTG
GCTAGTCACACCCTGTAGGCTCCTCTATATAACCCAGGGGCACAGGGGC
TGCCCTCATTCTACCACCACCTCCACAGCACAGACAGACACTCAGGAGC
CAGCCA

In certain embodiments, the CK8 promoter may comprise an additional C at the 5′ end and/or an additional dinucleotide GC at the 3′ end. The CK8 promoter comprises a 5′ end 130-bp enhancer element, followed by a 269-bp basal CK8 promoter, followed by a 48 bp or 50 bp MCK Exon 1 UTR sequence at the most 3′ end of the CK8 promoter. The 5′ end 130-bp enhancer element can be duplicated (e.g., having two tandem copies compared to one copy in CK8) to further enhancer transcription.

Thus in certain embodiments, the CK8 promoter is modified as a CK8e promoter (SEQ ID NO: 4), which comprises two copies of the 130-bp enhancer of SEQ ID NO: 5, the 269-bp fragment of the basal CK8 promoter of SEQ ID NO: 3 (SEQ ID NO: 6), and the 48-bp or 50-bp MCK exon 1 UTR region sequence (SEQ ID NO: 7 or 16).

(SEQ ID NO: 4)
TAGACTAGCATGCTGCCCATGTAAGGAGGCAAGGCCTGGGGACACCCGAGATGCCTGGTTATAATTAA
CCCAGACATGTGGCTGCCCCCCCCCCCCCAACACCTGCTGCCTCTAAAAATAACCCTGCATGTAGACT
AGCATGCTGCCCATGTAAGGAGGCAAGGCCTGGGGACACCCGAGATGCCTGGTTATAATTAACCCAGA
CATGTGGCTGCCCCCCCCCCCCCAACACCTGCTGCCTCTAAAAATAACCCTGCATGCCATGTTCCCGG
CGAAGGGCCAGCTGTCCCCCGCCAGCTAGACTCAGCACTTAGTTTAGGAACCAGTGAGCAAGTCAGCC
CTTGGGGCAGCCCATACAAGGCCATGGGGCTGGGCAAGCTGCACGCCTGGGTCCGGGGTGGGCACGGT
GCCCGGGCAACGAGCTGAAAGCTCATCTGCTCTCAGGGGCCCCTCCCTGGGGACAGCCCCTCCTGGCT
AGTCACACCCTGTAGGCTCCTCTATATAACCCAGGGGCACAGGGGCTGCCCTCATTCTACCACCACCT
CCACAGCACAGACAGACACTCAGGAGCCAGCCA
(SEQ ID NO: 5, 130-bp enhancer)
tagactagcatgctgcccatgtaaggaggcaaggcctggggacacccgagatgcctggttataattaa
cccagacatgtggctgcccccccccccccaacacctgctgcctctaaaaataaccctgcatg
(SEQ ID NO: 6, 269-bp basal CK8 promoter)
ccatgttcccggcgaagggccagctgtcccccgccagctagactcagcacttagtttaggaaccagtg
agcaagtcagcccttggggcagcccatacaaggccatggggctgggcaagctgcacgcctgggtccgg
ggtgggcacggtgcccgggcaacgagctgaaagctcatctgctctcaggggcccctccctggggacag
cccctcctggctagtcacaccctgtaggctcctctatataacccaggggcacaggggctgccctc
(UTR 48 bp MCK Exon 1, SEQ ID NO: 7)
attctaccaccacctccacagcacagacagacactcaggagccagcca.
(UTR 50 bp MCK Exon 1, SEQ ID NO: 16)
attctaccaccacctccacagcacagacagacactcaggagccagccagc

In certain embodiments, the muscle-specific control element comprises the nucleotide sequence of SEQ ID NO: 10 or SEQ ID NO: 11 of WO2017/181015.

| SEQ ID NO: 10 of WO2017/181015 (SEQ ID NO: 17): | |
|---|---|
| CAGCCACTAT GGGTCTAGGC TGCCCATGTA AGGAGGCAAG GCCTGGGGAC ACCCGAGATG | 60 |
| CCTGGTTATA ATTAACCCAG ACATGTGGCT GCTCCCCCCC CCCAACACCT GCTGCCTGAG | 120 |
| CCTCACCCCC ACCCCGGTGC CTGGGTCTTA GGCTCTGTAC ACCATGGAGG AGAAGCTCGC | 180 |
| TCTAAAAATA ACCCTGTCCC TGGTGG | 206 |
| SEQ ID NO: 11 of WO2017/181015 (SEQ ID NO: 18): | |
| GCTGTGGGGG ACTGAGGGCA GGCTGTAACA GGCTTGGGGG CCAGGGCTTA TACGTGCCTG | 60 |
| GGACTCCCAA AGTATTACTG TTCCATGTTC CCGGCGAAGG GCCAGCTGTC CCCCGCCAGC | 120 |
| TAGACTCAGC ACTTAGTTTA GGAACCAGTG AGCAAGTCAG CCCTTGGGGC AGCCCATACA | 180 |
| AGGCCATGGG GCTGGGCAAG CTGCACGCCT GGGTCCGGGG TGGGCACGGT GCCCGGGCAA | 240 |
| CGAGCTGAAA GCTCATCTGC TCTCAGGGGC CCCTCCCTGG GGACAGCCCC TCCTGGCTAG | 300 |
| TCACACCCTG TAGGCTCCTC TATATAACCC AGGGGCACAG GGGCTGCCCC CGGGTCAC | 358 |

In certain embodiments, the rAAV vectors of the invention can be operably linked to the muscle-specific control element comprising the MCK enhancer nucleotide sequence (see SEQ ID NO: 10 of WO2017/181015, incorporated herein by reference) and/or the MCK promoter sequence (see SEQ ID NO: 11 of WO2017/181015, incorporated herein by reference).

### 4. PolyA Signal Sequence, Introns, Exons, UTRs

In certain embodiments, the rAAV further comprises a polyadenylation (polyA) signal sequence for inserting a polyA sequence into a transcribed mRNA.

In certain embodiments, the polyA signal sequence is SEQ ID NO: 8, with the AATAAA sequence capitalized and double underlined:

(SEQ ID NO: 8)
AATAAAagatccttattttcattggatctgtgtgttggttttttgtgt

In certain embodiments, the polyA sequence is a 197-bp SV40 polyA signal sequence:

(SEQ ID NO: 9)
gatccagacatgataagatacattgatgagtttggacaaaccacaacta
gaatgcagtgaaaaaaatgctttatttgtgaaatttgtgatgctattgc
tttatttgtaaccattataagctgcAATAAAcaagttaacaacaacaa
ttgcattcattttatgtttcaggttcagggggaggtgtgggaggttttt
ta.

In certain embodiments, the polyA sequence is a 230-bp bGH polyA signal sequence: gtcgactagagctcgctgatcagcctcgactgtgccttctagttgccagccatctgttgtttgcccct

(SEQ ID NO: 10)
gtcgactagagctcgctgatcagcctcgactgtgccttctagttgccag
ccatctgttgtttgcccctcccccgtgccttccttgaccctggaaggtg
ccactcccactgtcctttcctAATAAAatgaggaaattgcatcgcatt
gtctgagtaggtgtcattctattctggggggtggggtggggcaggacag
caagggggaggattgggaagacaatagcaggcatg.

In certain embodiments, the polyA sequence is a 127-bp rBG polyA signal sequence:

(SEQ ID NO: 11)
gatctttttccctctgccaaaaattatggggacatcatgaagccccttg
agcatctgacttctggctAATAAAggaaatttattttcattgcaatag
tgtgttggaattttttgtgtctctcactcg.

### 5. AAV and Capsids

As used herein, the term “AAV” is a standard abbreviation for adeno-associated virus. Adeno-associated virus is a single-stranded DNA parvovirus that grows only in cells in which certain functions are provided by a co-infecting helper virus.

Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes.

The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and it encodes the three in-frame translated capsid proteins VP1, VP2, and VP3. Alternative splicing and non-consensus translational start sites are responsible for the production of the three related capsid proteins.

A single consensus polyadenylation site is located at map position 95 of the AAV genome. The life cycle and genetics of AAV are reviewed in Muzyczka, Current Topics in Microbiology and Immunology 158:97-129 (1992).

There are at least thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York) (incorporated herein by reference). However, it is fully expected that these same principles will be applicable to additional AAV serotypes since it is well known that the various serotypes are quite closely related, both structurally and functionally, even at the genetic level. See, for example, Blacklowe, 1988, pp. 165-174 of Parvoviruses and Human Disease, J. R. Pattison, ed.; and Rose, Comprehensive Virology 3: 1-61 (1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” or “(AAV) vector genome” as used herein interchangeably, refers to a vector comprising one or more polynucleotides of interest (or transgenes) that are flanked by AAV terminal repeat sequences (ITRs). Such AAV vectors can be replicated and packaged into infectious AAV viral particles when present in a host cell that has been transfected with a vector encoding and expressing rep and cap gene products.

Recombinant AAV vector genomes of the invention comprise nucleic acid molecule of the invention and one or more AAV ITRs flanking the nucleic acid molecule of the invention.

An “AAV virion” or “AAV viral particle” or “recombinant AAV (rAAV) viral particle” refers to a viral particle composed of at least one AAV capsid protein and an encapsidated polynucleotide AAV vector. If the particle comprises a heterologous polynucleotide (i.e., a polynucleotide other than a wild-type AAV genome such as the subject CpG reduced codon optimized microdystrophin coding sequence for delivery to a mammalian (muscle) cell), it is typically referred to as an “AAV vector/viral particle.” Thus, production of AAV viral particle necessarily includes production of AAV vector, as such a vector is contained within an AAV viral particle.

There are multiple serotypes of AAV, and the nucleotide sequences of the genomes of the AAV serotypes are known. For example, the nucleotide sequence of the AAV serotype 2 (AAV2) genome is presented in Srivastava et al., J Virol 45:555-564 (1983) as corrected by Ruffing et al., J Gen Virol 75:3385-3392 (1994). Both incorporated herein by reference. As other examples, the complete genome of AAV-1 is provided in GenBank Accession No. NC_002077 (incorporated herein by reference); the complete genome of AAV-3 is provided in GenBank Accession No. NC_001829 (incorporated herein by reference); the complete genome of AAV-4 is provided in GenBank Accession No. NC_001829 (incorporated herein by reference); the AAV-5 genome is provided in GenBank Accession No. AF085716 (incorporated herein by reference); the complete genome of AAV-6 is provided in GenBank Accession No. NC_001862 (incorporated herein by reference); at least portions of AAV-7 and AAV-8 genomes are provided in GenBank Accession Nos. AX753246 (incorporated herein by reference) and AX753249 (incorporated herein by reference), respectively (see also U.S. Pat. Nos. 7,282,199 and 7,790,449 relating to AAV-8); the AAV-9 genome is provided in Gao et al., J. Virol 78:6381-6388 (2004), incorporated herein by reference; the AAV-10 genome is provided in Mol. Ther. 13(1):67-76 (2006), incorporated herein by reference; and the AAV-11 genome is provided in Virology 330(2):375-383 (2004), incorporated herein by reference. The AAVrh74 serotype is described in Rodino-Klapac et al., J. Trans. Med. 5:45 (2007), incorporated herein by reference.

AAV DNA in the rAAV genomes may be from any AAV serotype for which a recombinant virus can be derived including, but not limited to, AAV serotypes AAV1, AAV2, AAV3A, AAV3B, AAV4, AAV5, AAV6, AAV7, AAV8, AAV9, AAV10, AAV11, AAV12, AAV13, AAVrh10, AAVrh74, AAVrh32, AAVrh34, and AAV-2i8.

In certain embodiments, to promote skeletal muscle specific expression, AAV1, AAV6, AAV8 or AAVrh.74 may be used.

In certain embodiments, the AAV has AAV9 serotype, or the capsid has the polypeptide of SEQ ID NO: 20 (AAV9 VP1):

(SEQ ID NO: 20)
MAADGYLPDWLEDNLSEGIREWWALKPGAPQPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWA
LPTYNNHLYKQISNSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSAHEGCLPPFPADVF
MIPQYGYLTLNDGSQAVGRSSFYCLEYFPSQMLRTGNNFQFSYEFENVPFHSSYAHSQSLDR
LMNPLIDQYLYYLSKTINGSGQNQQTLKFSVAGPSNMAVQGRNYIPGPSYRQQRVSTTVTQN
NNSEFAWPGASSWALNGRNSLMNPGPAMASHKEGEDRFFPLSGSLIFGKQGTGRDNVDADKV
MITNEEEIKITNPVATESYGQVATNHQSAQAQAQTGWVQNQGILPGMVWQDRDVYLQGPIWA
KIPHTDGNFHPSPLMGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRYLTRNL

Pseudotyped rAAV and production thereof are also suitable for the instant invention, and is disclosed in, for example, WO 01/83692 (incorporated herein by reference in its entirety).

Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In certain embodiments, the capsid is the SLB-101 capsid, which VP1 capsid has the sequence of SEQ ID NO: 21:

(SEQ ID NO: 21)
MAADGYLPDWLEDNLSEGIREWWALKPGAPQPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWA
LPTYNNHLYKQISNSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSAHEGCLPPFPADVF
MIPQYGYLTLNDGSQAVGRSSFYCLEYFPSQMLRTGNNFQFSYEFENVPFHSSYAHSQSLDR
LMNPLIDQYLYYLSKTINGSGQNQQTLKFSVAGPSNMAVQGRNYIPGPSYRQQRVSTTVTQN
NNSEFAWPGASSWALNGRNSLMNPGPAMASHKEGEDRFFPLSGSLIFGKQGTGRDNVDADKV
MITNEEEIKTTNPVATESYGQVATNHQSAQRGDLGLSAQAQTGWVQNQGILPGMVWQDRDVY
LQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL

### 6. Production of rAAV and Host Cells

The rAAV viral particles and vector genomes comprising the subject CpG depleted codon optimized microdystrophin coding sequence can be produced by any standard rAAV production methods, typically using a producer cell line.

General principles of rAAV production are reviewed in, for example, Carter, Current Opinions in Biotechnology 1533-1539, 1992; and Muzyczka, Curr. Topics in Microbial. and Immunol. 158:97-129, 1992). Various approaches are described in Ratschin et al., Mol. Cell. Biol. 4:2072, 1984; Hermonat et al., Proc. Natl. Acad. Sci. U.S.A. 81:6466, 1984; Tratschin et al., Mol. Cell. Biol. 5:3251, 1985; McLaughlin et al., J. Virol. 62: 1963, 1988; and Lebkowski et al., Mol. Cell. Biol. 7:349, 1988; Samulski et al., J. Virol. 63:3822-3828, 1989; U.S. Pat. No. 5,173,414; WO 95/13365, and corresponding U.S. Pat. No. 5,658,776; WO95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250, 1995; Paul et al., Human Gene Therapy 4:609-615, 1993; Clark et al., Gene Therapy 3:1124-1132, 1996; U.S. Pat. Nos. 5,786,211; 5,871,982; and 6,258,595. The foregoing documents are hereby incorporated by reference in their entirety herein, with particular emphasis on those sections of the documents relating to rAAV production.

Overall, a number of strategies differing in principles have been used for rAAV production, all of which can be used to produce the subject rAAV.

In certain embodiments, the subject rAAV is produced based on the helper-virus-free transient transfection method, with all cis and trans components (vector plasmid and packaging plasmids, along with helper genes isolated from adenovirus) in suitable host cells such as 293 cells. The transient-transfection method is simple in vector plasmid construction and generates high-titer AAV vectors that are free of adenovirus. The VP1 capsid proteins can be encoded by one of the plasmids used in transient transfection of the producer cell line.

Thus, in certain embodiments, the polynucleotide of the invention includes DNA plasmids comprising rAAV vector genomes of the invention. Such DNA plasmids can be used in the standard triple transfection method to produce rAAV. Specifically, DNA plasmids of the invention are transferred to cells permissible for infection with a helper virus of AAV (e.g., adenovirus, El-deleted adenovirus or herpes virus) for assembly of the rAAV vector genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV1, AAV2, AAV3A, AAV3B, AAV4, AAV5, AAV6, AAV7, AAV8, AAV9, AAV10, AAV11, AAV12, AAV13, AAVrh10, AAVrh.32, AAVrh34, or AAVrh.74. In certain embodiments, the capsid is a modified capsid such as SLB-101.

### Transient Transfection of Packaging Cell Line (HEK293)

In particular, in certain embodiments, the AAV vector is produced using transient transfection of a packaging cell line such as HEK293 cells. This is the most established AAV production method comprising plasmid transfection of human embryonic HEK293 cells. Typically, HEK293 cells are simultaneously transfected by a vector plasmid (containing the gene of interest, such as the subject polynucleotide encoding both the dystrophin minigene and the one or more additional coding sequences), and one or two helper plasmids, using calcium phosphate or polyethylenimine (PEI), a cationic polymer.

The helper plasmid(s) allow the expression of the four Rep proteins, the three AAV structural proteins VP1, VP2, and VP3, the AAP, and the adenoviral auxiliary functions E2A, E4, and VARNA. The additional adenoviral E1A/E1B co-factors necessary for rAAV replication are ex-pressed in HEK293 producer cells. Rep-cap and adenoviral helper sequences are either cloned on two separate plasmids or combined on one plasmid, hence both a triple plasmid system and a two plasmid system for transfection are possible. The triple plasmid protocol lends versatility with a cap gene that can easily be switched from one serotype to another.

The plasmids are usually produced by conventional techniques in E. coli using bacterial origin and anti-biotic-resistance gene or by minicircle technology.

Transient transfection in adherent HEK293 cells has been used for large-scale manufacturing of rAAV vectors. Recently, HEK293 cells have also been adapted to suspension conditions to be economically viable in the long term.

HEK293 lines are usually propagated in DMEM completed with L-glutamine, 5%-10% of fetal bovine serum (FBS), and 1% penicillin-streptomycin, except for suspension HEK293 cells that are maintained in serum-free suspension F17, Expi293, or other manufacturer-specific media. For adherent cells, the percentage of FBS can be reduced during AAV production in order to limit contamination by animal-derived components.

Generally, the rAAV vectors are recovered 48-72 hr after plasmid transfection from the cell pellet and/or supernatant, depending on the serotype.

Infection of Mammalian Cells with rHSV Vectors

HSV is a helper virus for replication of AAV in permissive cells. Thus, the HSV can serve both as a helper and as a shuttle to deliver the necessary AAV functions that support AAV genome replication and packaging to the producing cells.

AAV production based on co-infection with rHSV can efficiently generate a large amount of rAAV. In addition to high overall yields (up to 1.5×10⁵ vg/cell), the method is further advantageous in that it creates rAAV stocks with apparently increased quality as measured by an improved viral potency.

In this method, cells, typically the hamster BHK21 cell line or the HEK293 and derivatives, are infected with two rHSVs, one carrying the gene of interest bracketed by AAV ITR (rHSV-AAV), and the second with the AAV rep and cap ORFs of the desired serotype (rHSVrepcap). After 2-3 days, the cells and/or the media are collected, and rAAV is purified over multiple purification steps to remove cellular impurities, HSV-derived contaminants, and unpackaged AAV DNA.

Thus in some embodiments, HSV serves as a helper virus for AAV infection. In some embodiments, AAV growth is accomplished using non-replicating mutants of HSV with ICP27 deleted.

Certain methods for producing recombinant AAV viral particles in a mammalian cell have been known in the art and improved over the past decade. For example, U.S. Application Publication No. 20070202587 describes recombinant AAV production in mammalian cells based on co-infection of the cells with two or more replication-defective recombinant HSV vectors. U.S. Application Publication No. 20110229971 and Thomas et al. (Hum. Gene Ther. 20(8):861-870, 2009) describes a scalable recombinant AAV production method using recombinant HSV type 1 coinfection of suspension-adapted mammalian cells. Adamson-Small et al. (Hum. Gene Ther. Methods 28(1):1-14, 2017) describes an improved AAV production method in a serum-free suspension manufacturing platform using the HSV system.

In certain other embodiments, the subject rAAV is produced using a recombinant herpes simplex virus (rHSV)-based AAV production system, which utilizes rHSV vectors to bring the AAV vector and the Rep and Cap genes (i.e., the modified VP1 capsid gene of the invention) into the producer cells. The modified cap gene can be present in the rHSV vector that may also hosts the rAAV genome.

In certain embodiments, the AAV vectors of the invention are produced according to the method described in Adamson-Small et al. (Molecular Therapy—Methods & Clinical Development (2016) 3, 16031; doi:10.1038/mtm.2016.31, incorporated herein by reference), a scalable method for the production of high-titer and high quality adeno-associated type 9 vectors using the HSV platform. It is a complete herpes simplex virus (HSV)-based production and purification process capable of generating greater than 1×10¹⁴ rAAV9 vector genomes per 10-layer CellSTACK of HEK 293 producer cells, or greater than 1×10⁵ vector genome per cell, in a final, fully purified product. This represents a 5- to 10-fold increase over transfection-based methods. In addition, rAAV vectors produced by this method demonstrated improved biological characteristics when compared to transfection-based production, including increased infectivity as shown by higher transducing unit-to-vector genome ratios and decreased total capsid protein amounts, shown by lower empty-to-full ratios. This method can also be readily adapted to large-scale good laboratory practice (GLP) and good manufacturing practice (GMP) production of rAAV9 vectors to enable preclinical and clinical studies and provide a platform to build on toward late-phases and commercial production.

Infection of Insect Cells with Recombinant Baculovirus

In certain further embodiments, the subject rAAV is produced using a baculovirus system that requires simultaneous infection of insect cells with several baculovirus vectors to deliver the AAV vector cassette and the Rep and Cap genes (i.e., the modified VP1 capsid gene of the invention).

The baculovirus-Sf9 platform has been established as a GMP-compatible and scalable alternative AAV production method in mammalian cells. It can generate up to 2×10⁵ vector genomes (vg) per cell in crude harvests.

Current protocol involves infection of the Sf9 insect cells with two recombinant baculoviruses a baculovirus expression vector (BEV) allowing the synthesis of Rep78/52 and Caps, and a recombinant baculovirus carrying the gene of interest flanked by the AAV ITRs. Several serum-free media are adapted for Sf9 cell growth in suspension.

The dual-baculovirus-Sf9 production system has many advantages over other production platforms regarding these safety issues: (1) the use of serum-free media; (2) despite the discovery of adventitious virus transcripts in Sf cell lines, most of the viruses infecting insects do not replicate actively in mammalian cells; and (3) no helper virus is required for rAAV production in insect cells besides baculovirus.

In certain embodiments, stable Sf9 insect cell lines expressing Rep and Cap proteins are used, thus requiring the infection of only one recombinant baculovirus for the production of infectious rAAV vectors at high yield.

### Mammalian Stable Cell Lines.

The rAAV vectors can also be efficiently and scalably produced using stable mammalian producer cells stably expressing rep and cap genes. Such cells can be infected by wild-type Ad5 helper virus (which is genetically stable and can be easily produced at high titers) to induce high-level expression of rep and cap. Infectious rAAV vectors can be generated upon infection of these packaging cells lines with wild-type Ad type 5, and providing the rAAV genome by either plasmid transfection or after infection with a recombinant Ad/AAV hybrid virus.

Alternatively, Ad can be replaced by HSV-1 as the helper virus.

Suitable stable mammalian producer cells may include HeLa-derived producer cell lines, A549 cells, or HEK293 cells. A preferred HeLa cell line is HeLaS3 cells, a suspension adapted HeLa subclone.

The methods herein described can be used to manufacture the subject AAV vectors in animal components-free medium, preferably at 250-L scale, or 2,000-L commercial scale.

Regardless of how the rAAV viral particle of the invention is produced, the resulting rAAV may be purified by methods standard in the art such as by column chromatography or cesium chloride gradients. Methods for purifying rAAV vectors from helper virus are known in the art and include methods disclosed in, for example, Clark et al., Hum. Gene Ther. 10(6):1031-1039, 1999; Schenpp and Clark, Methods Mol. Med. 69:427-443, 2002; U.S. Pat. No. 6,566,118 and WO 98/09657.

The invention thus provides packaging/producer cells that produce infectious rAAV. In one embodiment, packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

In certain embodiments, the subject rAAV is produced based on certain AAV producer cell lines derived from, e.g., HeLa or A549 or HEK293 cells, which stably harbored AAV Rep/cap genes. The AAV vector cassette can either be stably integrated in the host genome or be introduced by an adenovirus that contained the cassette.

In certain embodiments, such producer cell line for rAAV production comprises an rAAV provirus that encodes the microdystrophin flanked by the AAV ITR sequences, wherein the rAAV provirus is integrated into the genome of the producer cell line for rAAV production.

A method of generating a packaging cell is to create a cell line that stably expresses all the necessary components for AAV particle production. For example, a plasmid (or multiple plasmids) comprising a rAAV vector genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. Sci. U.S.A. 79:2077-2081, 1982), addition of synthetic linkers containing restriction endonuclease cleavage sites (Laughlin et al., Gene 23:65-73, 1983) or by direct, blunt-end ligation (Senapathy & Carter, J. Biol. Chem. 259:4661-4666, 1984). The packaging cell line is then infected with a helper virus such as adenovirus. The advantages of this method are that the cells are selectable and are suitable for large-scale production of rAAV.

Other examples of suitable methods employ adenovirus or baculovirus rather than plasmids to introduce rAAV genomes and/or rep and cap genes into packaging cells.

Thus any of the packaging cells are within the scope of the host cell of the invention that comprise a polynucleotide, an AAV vector genome, or an AAV viral particle of the invention.

### 7. Treatment of Muscular Dystrophy Using rAAV

Another aspect of the invention provides a method of treating a muscular dystrophy in a human in need thereof, the method comprising administering to the human a therapeutically effective amount of the polynucleotide of the invention, the rAAV vector genome or the rAAV viral particle of the invention, or the pharmaceutical composition of the invention.

In certain embodiments, the muscular dystrophy is characterized by a loss-of-function a mutation in the dystrophin gene.

In certain embodiments, the muscular dystrophy is Duchenne muscular dystrophy (DMD), Becker muscular dystrophy (BMD), or X-linked dilated cardiomyopathy.

Thus a related aspect of the invention provides a method of treating muscular dystrophy (such as DMD and BMD) in a subject in need thereof, the method comprising administering to the subject a therapeutically effective amount of a recombinant AAV (rAAV) vector (such as one encapsidated in AAV9 or SLB-101 capsid) encoding a functional version of the gene defective in the muscular dystrophy, such as a microdystrophin gene, wherein the rAAV vector genome comprises any of the CpG reduced codon optimized polynucleotide of the invention (such as SEQ ID NO: 1).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the microdystrophin gene comprises a coding sequence for the microdystrophin protein of SEQ ID NO: 2, and the coding sequence comprises the nucleotide sequence of SEQ ID NO: 1, or a nucleotide sequence at least 70%, 75%, 80%, 85%, 90%, 95%, 96%, 97%, 98%, 99%, 99.2%, 99.4%, 99.6%, 99.8%, or 99.9% identical thereto. Optionally, the coding sequence is identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides, further optionally, the coding sequence substantially lacks CpG islands (e.g., no more than 1, 2, 3, 4, 5, 6, 7, 8, 9, or 10 CpG islands).

In certain embodiments, the method further comprises producing the subject rAAV prior to administering to the subject the rAAV so produced.

In any of the methods of the invention, the rAAV vector can be administered by intramuscular injection or intravenous injection.

In any of the methods of the invention, the rAAV vector or composition is administered systemically. For examples, the rAAV vector or composition is parentally administration by injection, infusion or implantation.

### 8. Pharmaceutical Composition and Uses Thereof

Another aspect of the invention provides a composition, such as a pharmaceutical composition, comprising any of the rAAV vectors, viral particle and vector genome comprising the CpG reduced codon optimized microdystrophin coding sequence of the invention.

In certain embodiments, the composition is a pharmaceutical composition, which may further comprise a therapeutically compatible carrier, excipient, diluents and/or adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

In another embodiment, the invention provides composition comprising any of the rAAV vectors, viral particle and vector genome comprising the CpG reduced codon optimized microdystrophin coding sequence of the invention for use in treating a subject suffering from dystrophinopathy or a muscular dystrophy, such as DMD or Becker Muscular dystrophy.

The compositions (e.g., pharmaceutical compositions) of the invention can be formulated for intramuscular injection or intravenous injection. The composition of the invention can also be formulated for systemic administration, such as parentally administration by injection, infusion or implantation. In addition, any of the compositions are formulated for administration to a subject suffering from dystrophinopathy or a muscular dystrophy, such as DMD, Becker muscular dystrophy or any other dystrophin associated muscular dystrophy.

In a further embodiment, the invention provides for use of any of the rAAV vectors, viral particle and vector genome comprising the CpG reduced codon optimized microdystrophin coding sequence of the invention for preparation of a medicament for reducing the subject suffering from dystrophinopathy or muscular dystrophy, such as DMD, Becker muscular dystrophy or any other dystrophin associated muscular dystrophy.

The invention contemplates use of the any of the rAAV vectors, viral particle and vector genome comprising the CpG reduced codon optimized microdystrophin coding sequence of the invention for the preparation of a medicament for administration to a patient diagnosed with DMD.

The invention also contemplates use of any of the rAAV vectors, viral particle and vector genome comprising the CpG reduced codon optimized microdystrophin coding sequence of the invention for the preparation of a medicament for administering any of the rAAV, viral particle and vector genome comprising the CpG reduced codon optimized microdystrophin coding sequence of the invention to a subject suffering from muscular dystrophy.

In any of the uses of the invention, the medicament can be formulated for intramuscular injection. In addition, any of the medicaments may be prepared for administration to a subject suffering from muscular dystrophy such as DMD or any other dystrophin associated muscular dystrophy.

### 9. Dosing and Administration

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, to about 1×10¹⁴ or more DNase resistant particles (DRP) per ml. Dosages may also be expressed in units of viral genomes (vg).

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

For administration, effective amounts and therapeutically effective amounts (also referred to herein as doses) may be initially estimated based on results from in vitro assays and/or animal model studies. For example, a dose may be formulated in animal models to achieve a circulating concentration range that includes the IC50 as determined in cell culture. Such information may be used to more accurately determine useful doses in subjects of interest.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the one or more coding sequences and/or micro-dystrophin.

Specifically, the formulations described herein may be administered by, without limitation, injection, infusion, perfusion, inhalation, lavage, and/or ingestion. Routes of administration may include, but are not limited to, intravenous, intradermal, intraarterial, intraperitoneal, intralesional, intracranial, intraarticular, intraprostatic, intrapleural, intratracheal, intranasal, intravitreal, intravaginal, intrarectal, topically, intratumoral, intramuscular, intravesicular, intrapericardial, intraumbilical, intraocularal, mucosal, oral, subcutaneous, and/or subconjunctival.

The invention provides for local administration or systemic administration of an effective dose of rAAV and compositions of the invention including combination therapy of the invention. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present invention may be accomplished by using any physical method that will transport the rAAV recombinant vector into the target tissue of an animal, such as the skeletal muscles. Administration according to the invention includes, but is not limited to, injection into muscle, the bloodstream and/or directly into the liver. Simply re-suspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

The dose of rAAV to be administered in methods disclosed herein will vary depending, for example, on the particular rAAV, the mode of administration, the treatment goal, the individual, and the cell type(s) being targeted, and may be determined by methods standard in the art.

The actual dose amount administered to a particular subject may also be determined by a physician, a veterinarian, or a researcher, taking into account parameters such as, but not limited to, physical and physiological factors including body weight, severity of condition, type of disease, previous or concurrent therapeutic interventions, idiopathy of the subject, and/or route of administration.

Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ vg, respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for tittering AAV are described in Clark et al., Hum. Gene Ther. 10:1031-1039, 1999.

Exemplary doses may range from about 1×10¹⁰ to about 1×10¹⁵ vector genomes (vg)/kilogram of body weight. In some embodiments, doses may comprise 1×10¹⁰ vg/kg of body weight, 1×10¹¹ vg/kg of body weight, 1×10¹² vg/kg of body weight, 1×10¹³ vg/kg of body weight, 1×10¹⁴ vg/kg of body weight, or 1×10¹⁵ vg/kg of body weight. Doses may comprise 1×10¹⁰ vg/kg/day, 1×10¹¹ vg/kg/day, 1×10¹² vg/kg/day, 1×10¹³ vg/kg/day, 1×10¹⁴ vg/kg/day, or 1×10¹⁵ vg/kg/day. Doses may range from 0.1 mg/kg/day to 5 mg/kg/day or from 0.5 mg/kg/day to 1 mg/kg/day or from 0.1 mg/kg/day to 5 μg/kg/day or from 0.5 mg/kg/day to 1 μg/kg/day. In other non-limiting examples, a dose may comprise 1 μg/kg/day, 5 μg/kg/day, 10 μg/kg/day, 50 μg/kg/day, 100 μg/kg/day, 200 μg/kg/day, 350 μg/kg/day, 500 μg/kg/day, 1 mg/kg/day, 5 mg/kg/day, 10 mg/kg/day, 50 mg/kg/day, 100 mg/kg/day, 200 mg/kg/day, 350 mg/kg/day, 500 mg/kg/day, or 1000 mg/kg/day. Therapeutically effective amounts may be achieved by administering single or multiple doses during the course of a treatment regimen (i.e., days, weeks, months, etc.).

In some embodiments, the pharmaceutical composition is in a dosage form of 10 mL of aqueous solution having at least 1.6×10¹³ vector genomes. In some embodiments, the dosage has a potency of at least 2×10¹² vector genomes per milliliter. In some embodiments, the dosage comprises a sterile aqueous solution comprising 10 mM L-histidine at pH 6.0, 150 mM sodium chloride, and 1 mM magnesium chloride. In some embodiments, the pharmaceutical composition is in a dosage form of 10 mL of a sterile aqueous solution comprising 10 mM L-histidine at pH 6.0, 150 mM sodium chloride, and 1 mM magnesium chloride; and having at least 1.6×10¹³ vector genomes.

In some embodiments, the pharmaceutical composition may be a dosage comprising between 1×10¹⁰ and 1×10¹⁵ vector genomes in 10 mL aqueous solution; between 1×10¹¹ and 1×10¹⁴ vector genomes in 10 mL aqueous solution; between 1×10¹² and 2×10¹³ vector genomes in 10 mL aqueous solution; or greater than or equal to about 1.6×10¹³ vector genomes in 10 mL aqueous solution. In some embodiments the aqueous solution is a sterile aqueous solution comprises about 10 mM L histidine pH 6.0, with 150 mM sodium chloride, and 1 mM magnesium chloride. In some embodiments, the dosage has a potency of greater than about 1×10¹¹ vector genomes per milliliter (vg/mL), greater than about 1×10¹² vg/mL, greater than about 2×10¹² vg/mL, greater than about 3×10¹² vg/mL, or greater than about 4×10¹² vg/mL.

In some embodiments, at least one AAV vector is provided as part of a pharmaceutical composition. The pharmaceutical composition may comprise, for example, at least 0.1% w/v of the AAV vector. In some other embodiments, the pharmaceutical composition may comprise between 2% to 75% of compound per weight of the pharmaceutical composition, or between 25% to 60% of compound per weight of the pharmaceutical composition.

In some embodiments, the dosage is in a kit. The kit may further include directions for use of the dosage.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

In some embodiments, for injection, formulations may be made as aqueous solutions, such as in buffers including, but not limited to, Hanks' solution, Ringer's solution, and/or physiological saline. The solutions may contain formulatory agents such as suspending, stabilizing, and/or dispersing agents. Alternatively, the formulation may be in lyophilized and/or powder form for constitution with a suitable vehicle control (e.g., sterile pyrogen-free water) before use.

Any formulation disclosed herein may advantageously comprise any other pharmaceutically acceptable carrier or carriers which comprise those that do not produce significantly adverse, allergic, or other untoward reactions that may outweigh the benefit of administration, whether for research, prophylactic, and/or therapeutic treatments. Exemplary pharmaceutically acceptable carriers and formulations are disclosed in Remington's Pharmaceutical Sciences, 18th Ed., Mack Printing Company, 1990, which is incorporated by reference herein for its teachings regarding the same. Moreover, formulations may be prepared to meet sterility, pyrogenicity, general safety, and purity standards as required by the United States FDA's Division of Biological Standards and Quality Control and/or other relevant U.S. and foreign regulatory agencies.

Exemplary, generally used pharmaceutically acceptable carriers may comprise, but are not limited to, bulking agents or fillers, solvents or co-solvents, dispersion media, coatings, surfactants, antioxidants (e.g., ascorbic acid, methionine, and vitamin E), preservatives, isotonic agents, absorption delaying agents, salts, stabilizers, buffering agents, chelating agents (e.g., EDTA), gels, binders, disintegration agents, and/or lubricants.

Exemplary buffering agents may comprise, but are not limited to, citrate buffers, succinate buffers, tartrate buffers, fumarate buffers, gluconate buffers, oxalate buffers, lactate buffers, acetate buffers, phosphate buffers, histidine buffers, and/or trimethylamine salts.

Exemplary preservatives may comprise, but are not limited to, phenol, benzyl alcohol, meta-cresol, methylparaben, propyl paraben, octadecyldimethylbenzyl ammonium chloride, benzalkonium halides, hexamethonium chloride, alkyl parabens (such as methyl or propyl paraben), catechol, resorcinol, cyclohexanol, and/or 3-pentanol.

Exemplary isotonic agents may comprise polyhydric sugar alcohols comprising, but not limited to, trihydric or higher sugar alcohols, (e.g., glycerin, erythritol, arabitol, xylitol, sorbitol, and/or mannitol).

Exemplary stabilizers may comprise, but are not limited to, organic sugars, polyhydric sugar alcohols, polyethylene glycol, sulfur-containing reducing agents, amino acids, low molecular weight polypeptides, proteins, immunoglobulins, hydrophilic polymers, and/or polysaccharides.

Formulations may also be depot preparations. In some embodiments, such long-acting formulations may be administered by, without limitation, implantation (e.g., subcutaneously or intramuscularly) or by intramuscular injection. Thus, for example, compounds may be formulated with suitable polymeric and/or hydrophobic materials (e.g., as an emulsion in an acceptable oil) or ion exchange resins, or as sparingly soluble derivatives (e.g., as a sparingly soluble salt).

Additionally, in various embodiments, the AAV vectors may be delivered using sustained-release systems, such as semipermeable matrices of solid polymers comprising the AAV vector. Various sustained-release materials have been established and are well known by those of ordinary skill in the art. Sustained-release capsules may, depending on their chemical nature, release the vector following administration for a few weeks up to over 100 days.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained co-expression of said one or more additional coding sequences and micro-dystrophin. The present invention thus provides methods of administering/delivering rAAV which co-expresses said one or more additional coding sequences and micro-dystrophin to an animal, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See Weintraub et al., Science 251:761-766, 1991), the myocyte-specific enhancer binding factor MEF-2 (Cserjesi and Olson, Mol Cell Biol 11:4854-4862, 1991), control elements derived from the human skeletal actin gene (Muscat et al., Mol Cell Biol 7:4089-4099, 1987), the cardiac actin gene, muscle creatine kinase sequence elements (Johnson et al., Mol Cell Biol 9:3393-3399, 1989), and the murine creatine kinase enhancer (mCK) element, control elements derived from the skeletal fast-twitch troponin C gene, slow-twitch cardiac troponin C gene and the slow-twitch troponin I gene: hypoxia-inducible nuclear factors (Semenza et al., Proc Natl Acad Sci U.S.A. 88:5680-5684, 1991), steroid-inducible elements and promoters including the glucocorticoid response element (GRE) (See Mader and White, Proc. Natl. Acad. Sci. U.S.A. 90:5603-5607, 1993), and other control elements.

Muscle tissue is an attractive target for in vivo DNA delivery, because it is not a vital organ and is easy to access. The invention contemplates sustained co-expression of miRNAs and micro-dystrophin from transduced myofibers.

As used herein, “muscle cell” or “muscle tissue” is meant a cell or group of cells derived from muscle of any kind (for example, skeletal muscle and smooth muscle, e.g., from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the one or more additional coding sequences and the coding region of the micro-dystrophin to a recipient cell either in vivo or in vitro, via a replication-deficient rAAV of the invention resulting in co-expression of the one or more additional coding sequences and micro-dystrophin by the recipient cell.

Thus, the invention provides methods of administering an effective dose (or doses, administered essentially simultaneously or doses given at intervals) of rAAV that encode said one or more additional coding sequences and micro-dystrophin to a patient in need thereof.

### EXAMPLES

### Example 1 Traditional Codon Optimization Introduced CpG Islands

There are various traditional codon optimization approaches, all aimed to improve expression level of coding sequences being codon optimized in a specific host cell. However, these approaches invariable introduce CpG islands during the process.

Using the online tool at the EBI website, EMBOSS Cpgplot, it is possible to predict the number and location of CpG islands in a particular input nucleotide sequence. The results of this analysis showed that all commonly used codon optimization approaches did introduce many CpG islands in their respective output sequences (i.e., codon optimized polynucleotides), while SEQ ID NO: 1, edited based on one of the codon optimized polynucleotides to remove CpG islands, no longer has predicted CpG islands by the online tool.

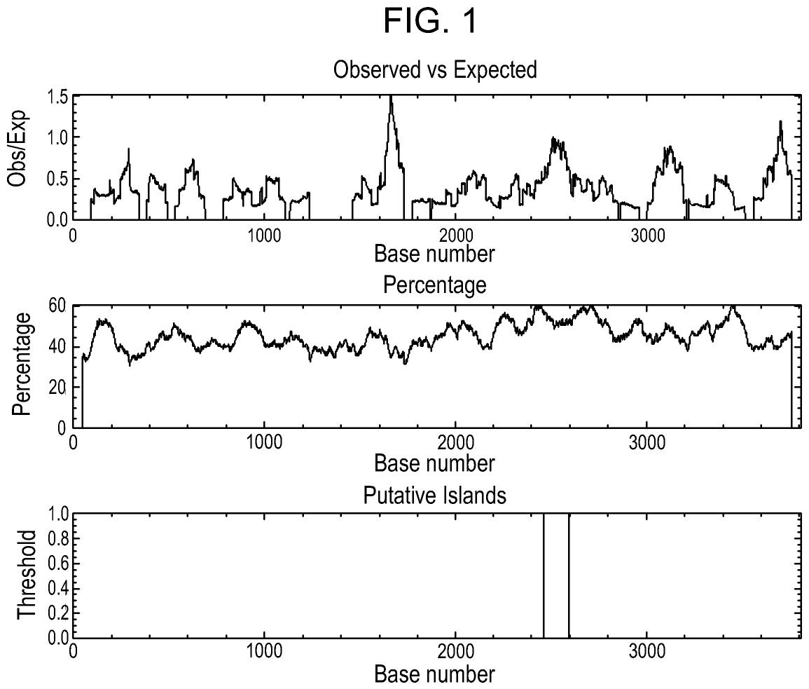

Specifically, the native, not codon optimized, microdystrophin coding sequence that encodes the microdystrophin of SEQ ID NO: 2, was analyzed using EMBOSS Cpgplot, using the parameters of: window size=100, minimum length=100, minimum observed=0.6, minimum percentage=50. The output of this analysis was shown in FIG. 1. It is apparent that the native human sequence has only one CpG island between nucleotides 2400-2500.

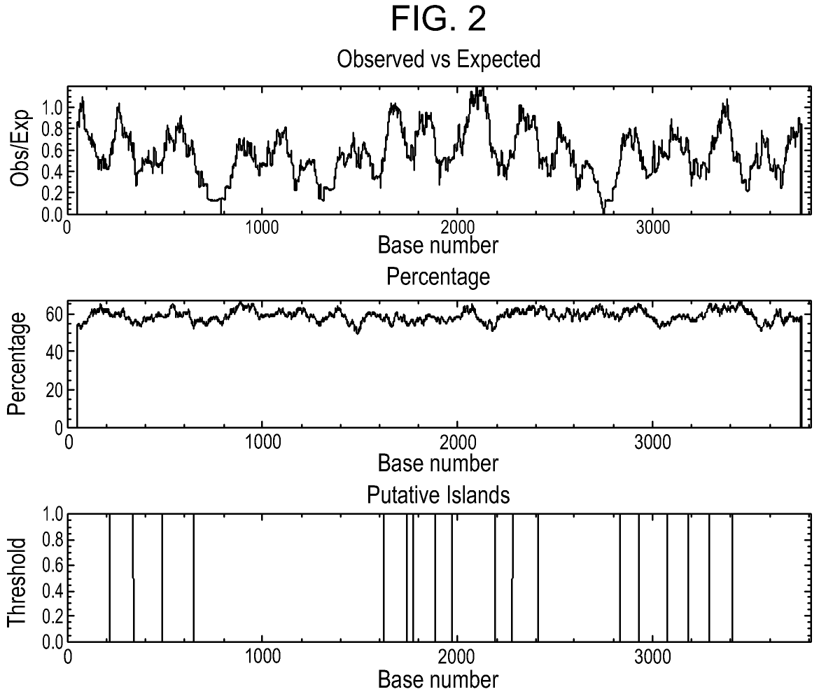

This native human MD5 coding sequence was then codon optimized using Gene Art, to generate a first codon optimized coding sequence for the same microdystrophin protein of SEQ ID NO: 2. EMBOSS Cpgplot identified nine CpG islands in this codon optimized sequence. See FIG. 2.

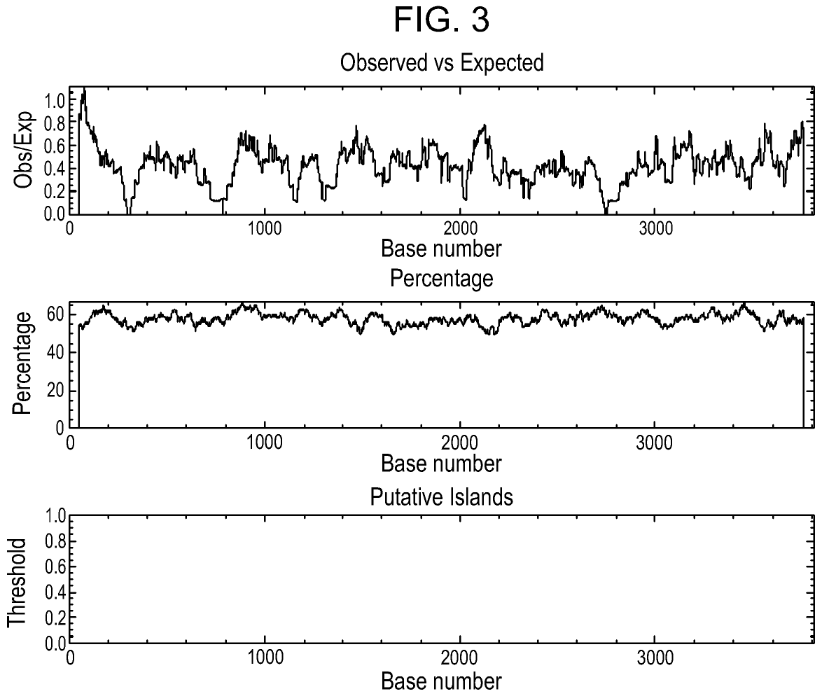

This first codon optimized coding sequence was modified by Applicant at the capitalized nucleotides in SEQ ID NO: 1, to arrive at SEQ ID NO: 1. EMBOSS Cpgplot identified no CpG islands in this codon optimized sequence. See FIG. 3.

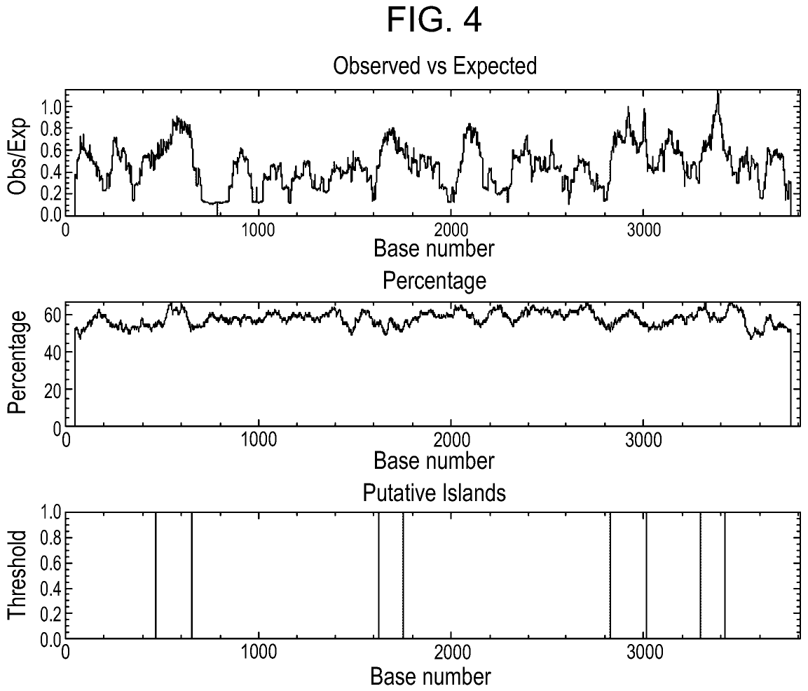

Next, GenScript was used to codon optimize the same native human MD5 to generate the second codon optimized coding sequence for SEQ ID NO: 2. EMBOSS Cpgplot identified four CpG islands in this codon optimized sequence. See FIG. 4.

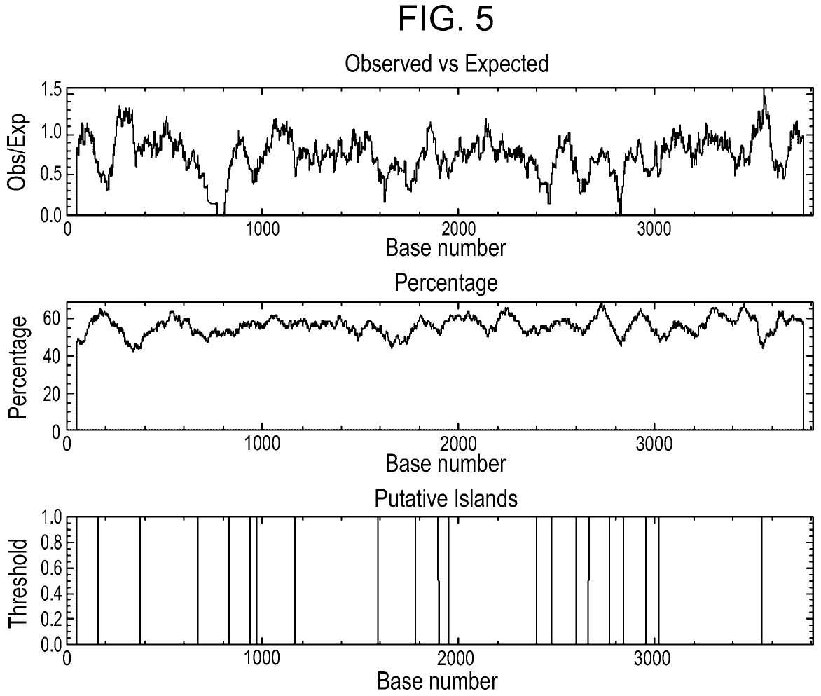

The same process was repeated for yet another codon optimization approach DNA2.0. EMBOSS Cpgplot identified eleven CpG islands in this codon optimized sequence. FIG. 5.

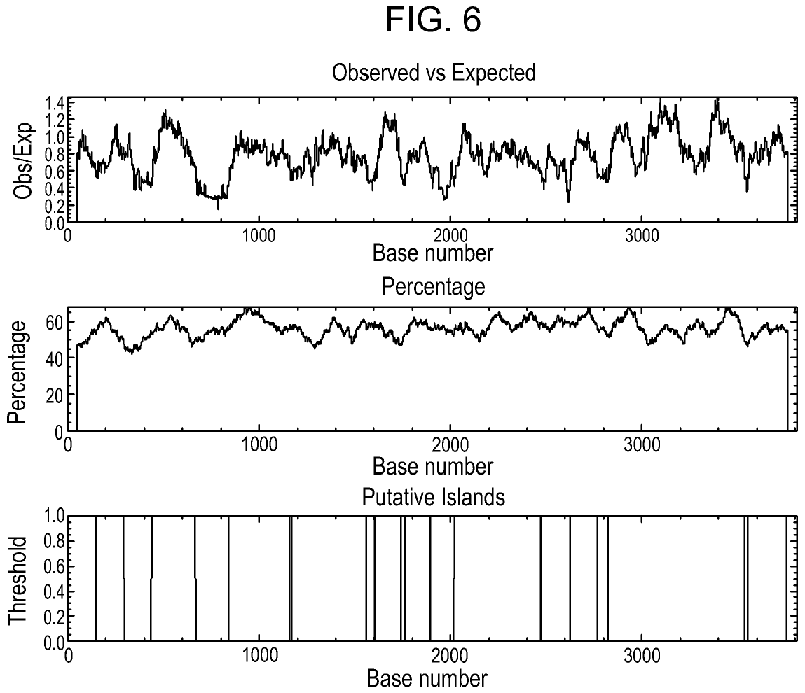

Interestingly, using the Gene Art codon-optimized coding sequence in FIG. 2 (having 9 CpG islands) as the input of a second round of codon optimization using DNA2.0 resulted in a coding sequence having 10 CpG islands, similar to the other DNA2.0 produced codon optimized sequence. See FIG. 6.

These data showed that traditional codon optimization approaches all tend to create CpG islands in the resulting codon optimized coding sequences. Multiple rounds of codon optimization using different approaches did not eliminate the CpG islands.

### Example 2 TLR9 Activation Assay

The CpG PAMP is recognized by the pattern recognition receptor (PRR) Toll-Like Receptor 9 (TLR9), which is constitutively expressed only in B cells and plasmacytoid dendritic cells (pDCs) in humans and other higher primates. Binding and activation of TLR9 by unmethylated CpG motifs promotes CTL responses to AAV vectors in non-clinical models.

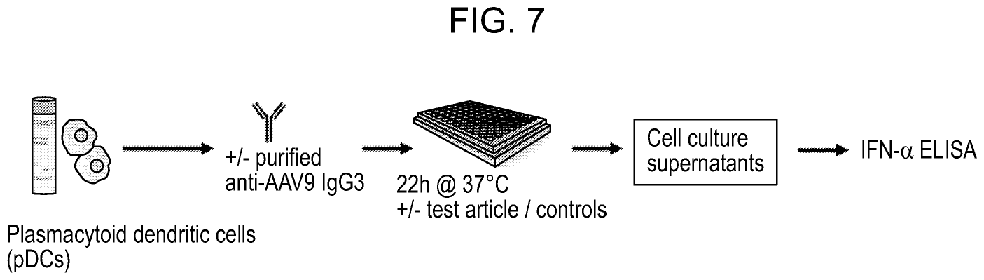

This assay, a schematic drawing is provided in FIG. 7, can be used to assess the potential and extent of a given polynucleotide coding sequence to trigger undesired host immune reaction due to the presence of CpG islands.

Briefly, human plasmacytoid dendritic cells (pDCs) isolated from a blood sample of a healthy donor, scheduled to receive a test polynucleotide having potential CpG islands, was purchased from STEMCELL Technologies. Cells were plated at 5×10⁴ (5E4) cells/well/100 μL cell culture medium in 96-well tissue culture plates. Anti-AAV capsid (e.g., anti-AAV9) IgG3 antibodies was added, followed by addition of test articles or vehicle controls. Tissue culture plates were incubated at 37° C. for about 22 hours. Cell culture supernatants were then collected, and the presence and amount of IFN-α as a readout of TLR9 activation was measured in ELISA.

Using this assay, the AAV9 viral particles encapsidating a vector genome comprising the Green Fluorescent Protein (GFP) was shown to increase TLR9 dependent IFN-α production (data not shown). In addition, empty AAV9 capsid without encapsidated vector genome did not trigger TLR9 activation. Therefore, this assay can be utilized to investigate innate immune response to AAV9 viral particles encapsidating the vector genome comprising modified CpG island.

1. A polynucleotide encoding the microdystrophin of SEQ ID NO: 2, said polynucleotide comprising the nucleotide sequence of SEQ ID NO: 1, or a nucleotide sequence at least 70%, 75%, 80%, 85%, 90%, 95%, 96%, 97%, 98%, 99%, 99.2%, 99.4%, 99.6%, 99.8%, or 99.9% identical thereto.

2. The polynucleotide of claim 1, which is identical to SEQ ID NO: 1 at each capitalized nucleotides, or differ by no more than 1, 2, 3, 4, 5, 6, 7, 8, 9, 10, 11, 12, 13, 14, 15, 16, 17, 18, 19, or 20 capitalized nucleotides.

3. The polynucleotide of claim 1 or 2, which substantially lacks CpG islands (e.g., no more than 1, 2, 3, 4, 5, 6, 7, 8, 9, or 10 CpG islands based on EMBOSS Cpgplot analysis).

4. The polynucleotide of any one of claims 1-3, comprising, consisting essentially of, or consisting of a nucleotide sequence at least 95% identical to SEQ ID NO: 1.

5. The polynucleotide of any one of claims 1-3, comprising, consisting essentially of, or consisting of a nucleotide sequence at least 97% identical to SEQ ID NO: 1.

6. The polynucleotide of any one of claims 1-3, comprising, consisting essentially of, or consisting of a nucleotide sequence at least 99% identical to SEQ ID NO: 1.

7. The polynucleotide of claim 1, comprising the nucleotide sequence of SEQ ID NO: 1.

8. The polynucleotide of claim 1, consisting of the nucleotide sequence of SEQ ID NO: 1.

9. An adeno associated virus (AAV) vector genome, comprising the polynucleotide of any one of claims 1-8, wherein the AAV vector genome is capable of being packaged inside an AAV capsid.

10. A recombinant adeno associated viral (rAAV) particle, comprising an AAV capsid, and an AAV vector genome comprising the polynucleotide of any one of claims 1-8, wherein the AAV vector genome is encapsidated within the AAV capsid.

11. The AAV vector genome of claim 9 or the rAAV viral particle of claim 10, wherein the polynucleotide is operably linked to a transcriptional regulatory element.

12. The AAV vector genome or the rAAV viral particle of claim 11, wherein the transcriptional regulatory element comprises a promoter.

13. The AAV vector genome or the rAAV viral particle of claim 12, wherein the promoter is a muscle-specific promoter.

14. The AAV vector genome or the rAAV viral particle of claim 13, wherein the muscle-specific promoter is CK8 promoter, cardiac troponin T (cTnT) promoter, CK7 promoter, CK9 promoter, truncated MCK (tMCK), myosin heavy chain (MHC) promoter, hybrid α-myosin heavy chain enhancer-/MCK enhancer-promoter (MHCK7), a muscle specific creatine kinase (MCK) promoter, human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

15. The AAV vector genome or the rAAV viral particle of claim 13, wherein the muscle-specific promoter is a CK8 promoter; optionally, said CK8 promoter comprises the nucleotide sequence of SEQ ID NO: 3 or 4.

16. The AAV vector genome or the rAAV viral particle of any one of claims 9-15, wherein the vector genome further comprises a polyadenylation signal sequence, such as the polyA signal sequence of SEQ ID NO: 8.

17. The AAV vector genome or the rAAV viral particle of claim 16, wherein the polyadenylation signal sequence comprises an SV40 polyadenylation signal sequence (e.g., SEQ ID NO: 9), a bovine growth hormone (bGH) polyadenylation signal sequence (e.g., SEQ ID NO: 10), or a rabbit beta globin (rBG) polyadenylation signal sequence (e.g., SEQ ID NO: 11).

18. The AAV vector genome or the rAAV viral particle of any one of claims 9-17, wherein the vector genome further comprises a 3′ ITR sequence, such as an AAV2 3′ ITR sequence.

19. The AAV vector genome or the rAAV viral particle of any one of claims 9-18, wherein the vector genome further comprises a 5′ ITR sequence, such as an AAV2 5′ ITR sequence.

20. The AAV vector genome or the rAAV viral particle of claim 18 or 19, wherein the 5′ ITR sequence, and/or the 3′ ITR sequence (1) comprise or are SEQ ID NOs: 12 and 13, respectively; or (2) comprise or are SEQ ID NOs: 24 and 27, respectively.

21. The AAV vector genome or the rAAV viral particle of any one of claims 9-20, wherein the vector genome further comprises an intron and/or an exon sequence that enhances expression of the microdystrophin.

22. The AAV vector genome or the rAAV viral particle of claim 21, wherein the intron comprises SEQ ID NO: 14.

23. The AAV vector genome or the rAAV viral particle of any one of claims 9-20, wherein the vector genome further comprises a 5′ UTR sequence, and/or a 3′ UTR sequence.

24. The AAV vector genome or the rAAV viral particle of any one of claims 9-23, comprising, consisting essentially of, or consisting of the nucleotide sequence of SEQ ID NO: 15, or a nucleotide sequence at least 90%, 95%, 96%, 97%, 98%, 99%, 99.5%, 99.7% or 99.9% identical thereto.

25. The rAAV viral particle of any one of claims 10-24, wherein the capsid is of the serotype of SLB-101, AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, or AAVhu37.

26. The rAAV viral particle of any one of claims 10-24, wherein the capsid is of the serotype of SLB-101 or AAV9.

27. A recombinant adeno-associated virus (rAAV) viral particle, comprising an SLB-101 or AAV9 capsid, and a vector genome encapsidated therein, wherein said vector genome comprises a polynucleotide sequence encoding the MD5 microdystrophin of SEQ ID NO: 2.

28. The rAAV viral particle of claim 27, wherein the polynucleotide sequence comprises the nucleotide sequence of SEQ ID NO: 1.

29. The rAAV viral particle of claim 27, wherein the polynucleotide sequence comprises a nucleotide sequence at least 95%, 96%, 97%, 98%, 99% identical to SEQ ID NO: 1, and is identical to SEQ ID NO: 1 at each capitalized nucleotides.

30. The rAAV viral particle of any one of claims 27-29, wherein said vector genome comprises a muscle-specific control element operably linked to the polynucleotide sequence.

31. The rAAV viral particle of claim 30, wherein said muscle-specific control element comprises a CK8 promoter, such as the CK8 promoter of the nucleotide sequence of SEQ ID NO: 3 or 4.

32. The AAV viral particle of any one of claims 27-31, wherein the vector genome further comprises a polyadenylation signal sequence, such as a polyA signal sequence comprising SEQ ID NO: 8.

33. The AAV viral particle of claim 32, wherein the polyadenylation signal sequence comprises an SV40 polyadenylation signal sequence (SEQ ID NO: 9), a bovine growth hormone (bGH) polyadenylation signal sequence (SEQ ID NO: 10), or a rabbit beta globin (rBG) polyadenylation signal sequence (SEQ ID NO: 11).

34. The AAV viral particle of any one of claims 27-33, wherein the vector genome further comprises a 3′ ITR sequence, such as SEQ ID NO: 3′ ITR; and a 5′ ITR sequence, such as SEQ ID NO: 5′ ITR.

35. The AAV viral particle of any one of claims 27-34, comprising, consisting essentially of, or consisting of the nucleotide sequence of SEQ ID NO: 15, or a nucleotide sequence at least 90%, 95%, 96%, 97%, 98%, 99%, 99.5%, 99.7% or 99.9% identical thereto.

36. A pharmaceutical composition comprising the polynucleotide of any one of claims 1-8, the rAAV vector genome or the rAAV viral particle of any one of claims 9-35, and a pharmaceutically acceptable carrier.

37. The pharmaceutical composition of claim 36, which is suitable or formulated for intravenous, subcutaneous, intramuscular, intradermal, intraperitoneal, or intrathecal administration.

38. A method of treating a muscular dystrophy in a human in need thereof, the method comprising administering to the human a therapeutically effective amount of the polynucleotide of any one of claims 1-8, the rAAV vector genome or the rAAV viral particle of any one of claims 9-35, or the pharmaceutical composition of claim 36-37.

39. The method of claim 37, wherein the muscular dystrophy is characterized by a loss-of-function a mutation in the dystrophin gene.

40. The method of claim 38 or 39, wherein the muscular dystrophy is Duchenne muscular dystrophy (DMD), Becker muscular dystrophy (BMD), or X-linked dilated cardiomyopathy.

41. The method of any one of claims 38-40, wherein the rAAV viral particle is administered at a dose of about 1×10¹² to about 1×10¹⁶ vector genome (vg)/kg, or about 1×10¹³ to about 1×10¹⁵ vector genome (vg)/kg.

42. A host cell comprising the polynucleotide of any one of claims 1-8, or the rAAV vector genome or the rAAV viral particle of any one of claims 9-35.

43. The host cell of claim 42, which is a HeLa cell, a Cos7 cell, a HEK293 cell, an A549 cell, a BHK cell, a Vero cell, an RD cell, an HT-1080 cell, an ARPE-19 cell, or a MRC-5 cell.

44. The host cell of claim 43, wherein the host cell is a HeLa cell or a 293/293T cell.
